(19) United States
(12) Patent Application Publication
(10) Pub. No.: US 2009/0181369 A1
(43) Pub. Date: Jul. 16, 2009

(54) Methods for the Treatment of Disease

(76) Inventors: George Q Daley, Weston, MA (US); Tal Raz, Brookline, MA (US); Mohammad Azam, Boston, MA (US)

Correspondence Address: DAVID S. RESNICK, NIXON PEABODY LLP, 100 SUMMER STREET, BOSTON, MA, 02110-2131, US

(21) Appl. No.: 11/921,096

(22) PCT Filed: May 30, 2006
(86) PCT No.: PCT/US2006/020933

§ 371 Date: May 29, 2008

Related U.S. Application Data
(60) Provisional application No. 60/685,666, filed on May 27, 2005.

(51) Int. Cl. C12Q 1/68; C12Q 1/48

(52) U.S. Cl. 435/6; 435/15

(57) ABSTRACT

The present invention is directed to methods to determine the likelihood of therapeutic effectiveness of a farnesyl transferase inhibitor (FTI). The method comprises determining whether the gene encoding the farnesyl transferase beta subunit (FNTB) of said patient comprises at least one nucleic acid variance that causes an alteration in an amino acid residue. The change in the amino acid residue is associated with resistance to a FTI. The absence of at least one variance indicates that the FTI is likely to be effective.

### CROSS REFERENCE

This application is an International Application, which claims priority benefit of U.S. Provisional Application Ser. No. 60/685,666, filed on May 27, 2005, the content of which is relied upon and incorporated herein by reference in its entirety, and benefit priority under 35 U.S.C. §119(e).

This invention was made with Government Support under Contract Nos. F32 CA101505-01 and R01 CA86991, awarded by the National Institutes of Health. The Government has certain rights in the invention.

### BACKGROUND OF THE INVENTION

Cancer remains a major health concern. Despite increased understanding of many aspects of cancer; the methods available for its treatment continue to have limited success. First of all, the number of cancer therapies is limited, and none provides an absolute guarantee of success. Second, there are many types of malignancies, and the success of a particular therapy for treating one type of cancer does not mean that it will be broadly applicable to other types. Third, many cancer treatments are associated with toxic side effects. Most treatments rely on an approach that involves killing off rapidly growing cells; however, these treatments are not specific to cancer cells and can adversely affect any dividing healthy cells. Fourth, assessing molecular changes associated with cancerous cells remains difficult. Given these limitations in the current arsenal of anti-cancer treatments, there remains a pressing need for improved therapeutic agents that are specifically targeted to the critical genetic lesions that direct tumor growth.

The clinical development of rationally designed, narrowly targeted, cancer therapy against tyrosine kinases (such as Her2/Neu, BCR/ABL, EGFR, and others) has shown great promise and resulted in the FDA approval of a number of drugs. The most dramatic clinical success resulted from the treatment of chronic myeloid leukemia (CML) patients with the BCR/ABL inhibitor imatinib, resulting in a response rate that is well over 90% in chronic phase patients¹. Imatinib response in CML patients has been thoroughly studied in the past number of years, and it is now well documented that although response is durable in patients treated at the chronic phase of the disease, it is invariably transient in patients treated at the advanced stages. This drug resistance is mainly due to the development of mutations in the BCR/ABL target protein. A number of second generation compounds designed to target mutant forms of BCR/ABL known to cause imatinib resistance are currently under development. Clinical trials using these new agents are underway, and early reports of trial outcomes show great promise². To date, the clinical development of rational, target specific cancer therapy has focused on tyrosine kinase proteins. However, non-kinase signal transduction targets are being investigated as well. A major focus in the clinical development of non-kinase inhibitors is the farnesyl transferase protein (FTase).

FTase is responsible for the post translational prenylation required for the activation of a number of proteins acting in signal transduction pathways. FTase attaches a lipid moiety to the C terminus of its substrate proteins. This prenylation was reported to be necessary for the activity of proteins such as Ras, Rheb, CENP-E, and others³⁻⁵. The farnesyltransferase inhibitors (FTIs) are currently being evaluated in clinical trials against both solid tumors and hematopoietic malignancies. A number of different agents are under clinical investigation including lonafarnib, tipifarnib, and BMS214662. Moderate activity has been reported in phase I and II trials using FTIs as monotherapy (⁶⁻⁸). Recently, the focus of clinical trials has shifted to the use of combination therapy, based on successful pre-clinical models (e.g. ⁹⁻¹²). Promising results have been published for using FTIs in combination with imatinib for the treatment of CML, and in combination with taxanes for the treatment of breast cancer (reviewed in^{13,14}). The reason for the tendency of FTIs to act synergistically with other agents is not well understood and may be due to FTIs' inhibition of a number of signal transduction proteins.

In addition to FTIs activity against cancer, preclinical results were published on the sensitivity of a number of eukaryotic pathogens (e.g. P. falciparum and T. brucei) to FTase inhibition (reviewed in ¹⁵) Reports have also been published on the possibility of administering FTIs to patients with the Hutchinson-Giford progeria syndrome (HGPS). It has been suggested that this syndrome is caused by the accumulation of farnesylated prelamin A in the cell's nucleus resulting in misshapen nuclei. Recently a number of studies have reported correction of this phenotype in mouse and patient cells by administration of FTIs in cell culture¹⁶⁻¹⁸.

A significant limitation in using these compounds is that recipients thereof may develop a resistance to their therapeutic effects after they initially respond to therapy, or they may not respond to FTIs to any measurable degree ab initio. In fact, one such resistance-conferring mutation to 2 tricyclic FTIs (developed by Schering Plough Corporation) has already been described in vitro²⁹. Thus, although the compounds may, at first, exhibit strong anti-tumor properties, they may soon become less potent or entirely ineffective in the treatment of cancer. Moreover, since medical research has heretofore not elucidated the biomolecular or pathological mechanism responsible for this resistance, patients who have exhibited such resistance to date have been left with few therapeutic alternatives to treat their disease. For patients that develop resistance, this potentially life-saving therapeutic mechanism did not achieve what they had hoped for and so desperately needed—an active therapy for cancer.

Accordingly there is a need to improve the therapeutic potential of FTIs in the treatment of cancer, including by identifying resistance-conferring mutations in their target proteins. There is a significant need in the art for a satisfactory treatment of cancer, and specifically leukemias, specifically to treat, which incorporates the benefits of FTI therapy, while obviating the resistance developed in response thereto by many patients, and overcoming the non-responsiveness exhibited by still other patients. Such a treatment could have a dramatic impact on the health of individuals.

### SUMMARY OF THE INVENTION

We have surprisingly discovered that the presence of specific mutations in the gene encoding the beta subunit of farnesyl transferase (FNTB) confer resistance to the FTI lonafarnib. We have also discovered that certain patients resistant to lonafarnib carry the same mutations in their gene(s) encoding FNTB. Thus, patients having these mutations will be less responsive to FTI therapy, for example lonafarnib.

Accordingly, the present invention provides a novel method to determine the likelihood of therapeutic effectiveness of a farnesyl transferase inhibitor (FTI) in a patient. In one embodiment, the patient is affected with cancer. The method comprises determining whether the gene encoding the target farnesyl transferase beta subunit (FNTB) comprises at least one nucleic acid variance that causes an alteration in an amino acid residue, where the change in the amino acid residue is associated with resistance to a FTI. The absence of at least one variance indicates that the FTI is likely to be effective. The patient's therapeutic regimen can then be designed to reflect the likely effectiveness of the FTI. Preferably, the farnesyl transferase inhibitor is lonafarnib (SCH66336), tipifarnib (R115777), L-778,123, or BMS214662. In one preferred embodiment, lonafarnib.

### BRIEF DESCRIPTION OF THE DRAWINGS

FIGS. 1A-1B show that lonafarnib resistance of each mutant was verified by two assays. FIG. 1A shows a soft agar plating assay where cells were plated in the presence of varying lonafarnib concentrations and allowed to proliferate for 14 days. Drug resistance was measured as a ratio between the number of colonies formed in drug to the number of colonies formed in diluent alone. FIG. 1B shows a western blot analysis of mutation harboring cells grown in varying drug concentrations. Protein farnesylation is visualized by western blot since farnesylated proteins have a faster migration on a gel than unfarnesylated ones.

FIG. 2 shows the strategy used for determining FNTB variants demonstrating resistance to farnesyl transferase inhibitors.

FIG. 3 shows the effect of lonafarnib and imatinib drug combination on BaF3 cells harboring G250E and M351T mutations.

### DETAILED DESCRIPTION OF THE INVENTION

We have surprisingly discovered that the presence of specific mutations in the gene encoding the beta subunit of farnesyl transferase (FNTB) confer resistance to the FTI lonafarnib. We have also discovered that certain patients resistant to lonafarnib carry the same mutations in their gene(s) encoding FNTB. Thus, patients having these mutations will be less responsive to FTI therapy, for example lonafarnib.

Accordingly, the present invention provides a novel method to determine the likelihood of therapeutic effectiveness of a farnesyl transferase inhibitor (FTI) in a patient. In one embodiment, the patient is affected with cancer. The method comprises determining whether the gene encoding the farnesyl transferase beta subunit (FNTB) of said patient comprises at least one nucleic acid variance that causes an alteration in an amino acid residue, where the change in the amino acid residue is associated with resistance to a FTI. The absence of at least one variance indicates that the FTI is likely to be effective. The patient's therapeutic regimen can then be designed to reflect the likely effectiveness of the FTI.

Preferably, the farnesyl transferase inhibitor is lonafarnib (SCH66336), tipifarnib (R115777), L-778,123, or BMS21466. In one preferred embodiment, lonafarnib.

Preferably, the amino acid residue that is altered in the variant FNTB is one or more of the following residues: C95, W106, I107, P152, A155, V195, G196, L213, G224, G241, V242, E265, M282, E285, A305, F360, and Y361. In one preferred embodiment, the altered amino acid residue is one or more of the following residues: C95, W106, I107, P152, A155, G241, V242, and Y361. In one embodiment, the altered amino acid residue is not P152. In one embodiment, the alerted amino acid residue is not Y361. In one embodiment, the alerted amino acid residue is not Y365. In one embodiment, the alerted amino acid residue is not R202. In one embodiment, the altered amino acid residue is one or more of the following mutations: C95R, W106R, I107V, P152S, A155S, V195D, G196R, L213P, G224S, G241E, V242I, E265K, M282V, E285K, A305T, F360S, Y361L, Y361S, and Y361H. In one preferred embodiment, the altered amino acid residue is one or more of the following mutations: C95R, W106R, I107V, P152S, A155S, G241E, V242I, Y361S, and Y361H. In one embodiment, the altered amino acid residue is not the mutation Y361L. In one embodiment, the altered amino acid residue is not the mutation Y361M. In one embodiment, the altered amino acid residue is not the mutation Y361I. In one embodiment, the altered amino acid residue is not the mutation Y361C. In one embodiment, the altered amino acid residue is not the mutation P152M.

### Inhibitors of Farnesyl Protein Transferases

Farnesyl protein transferase inhibitors, also referred to herein as FTIs, are well known in the art. Any inhibitor of a farnesyl transferase can be used in the methods of the present invention. Preferred FTIs include but are not limited to Lonafarnib, also known as SCH66336 (CAS-193275-84-2; (+)-4-[2-[4-(8-Chloro-3,10-dibromo-6,11-dihydro-5H-benzo[5,6]cyclohepta[1,2-b]-pyridin-11(R)-yl)-1-piperidinyl]-2-oxo-ethyl]-1-piperidinecarboxamide), tipifarnib, also known as Zarnestra or R115777 (14C-labeled (R)-6-[amino(4-chlorophenyl)(1-methyl-1H-imidazol-5-yl)methyl]-4-(3-chlorophenyl)-1-methyl-2(1H)-quinolinone), L-778,123 (Merck), or BMS214662 ((R)-7-cyano-2,3,4,5-tetrahydro-1-(1H-imidazol-4-ylmethyl)-3-(phenylmethyl)-4-(2-thienyl sulfonyl)-1H-1,4-benzodiazepine). In one particularly preferred embodiment, the FTI is Lonafarnib.

“Farnesyl transferase inhibitors” as used herein refers to any compound or agent that is capable of inhibiting a farnesyl protein transferase's ability to transfer of farnesol to a protein or peptide having a farnesyl acceptor moiety. As used herein, the phrase “capable of catalyzing the transfer of farnesol to a protein or peptide having a farnesyl acceptor moiety,” is intended to refer to the functional attributes of farnesyl transferase enzymes of the present invention, which catalyze the transfer of farnesol, typically in the form of all-trans farnesol, from all-trans farnesyl pyrophosphate to proteins which have a sequence recognized by the enzyme for attachment of the farnesyl moieties. Thus, the term “farnesyl acceptor moiety” is intended to refer to any sequence, typically a short amino acid recognition sequence, which is recognized by the enzyme and to which a farnesyl group will be attached by such an enzyme.

Farnesyl acceptor moieties have been characterized by others in various proteins as a four amino acid sequence found at the carboxy terminus of target proteins. This four amino acid sequence has been characterized as —C-A-A-X, wherein “C” is a cysteine residue, “A” refers to any aliphatic amino acid, and “X” refers to any amino acid. Of course, the term “aliphatic amino acid” is well-known in the art to mean any amino acid having an aliphatic side chain, such as, for example, leucine, isoleucine, alanine, methionine, valine, etc. While the most preferred aliphatic amino acids, for the purposes of the present invention include valine and isoleucine, it is believed that virtually any aliphatic amino acids in the designated position can be recognized within the farnesyl acceptor moiety. In addition, the enzyme has been shown to recognize a peptide containing a hydroxylated amino acid (serine) in place of an aliphatic amino acid (CSIM). Of course, principal examples of proteins or peptides having a farnesyl acceptor moiety, for the purposes of the present invention, will be the p21^{rax} proteins, including p21^{H-ras} p21^{K-rasA}, p21^{rasB} and p21^{N-ra}. Thus, in light of the present disclosure, a wide variety of peptidyl sequences having a farnesyl acceptor moiety will become apparent.

Other farnesyl transferase inhibitors that can be used in the methods of the present invention include those disclosed for example in U.S. Patent Application Publication Nos. 20040157773, 20040110769, and 20040044032.

In one embodiment of the invention, the farnesyl transferase inhibitor of the present invention is a peptide, such as those peptides described in U.S. Patent Application No. 20030170766. Such peptide inhibitors can include a farnesyl acceptor or inhibitory amino acid sequence having the amino acids —C-A-A-X, wherein: C=cysteine; A=any aliphatic, aromatic or hydroxy amino acid; and X=any amino acid. Typically, the farnesyl acceptor or inhibitory amino acid sequence will be positioned at the carboxy terminus of the protein or peptide such that the cysteine residue is in the fourth position from the carboxy terminus. In preferred embodiments, the inhibitor will be a relatively short peptide such as a peptide from about 4 to about 10 amino acids in length. For example, one inhibitor can be a tetrapeptide which incorporates the —C-A-A-X recognition structure. Shorter peptides can also be used.

While, broadly speaking, it is believed that compounds exhibiting an IC₅₀ of between about 0.01.mu.M and 10.mu.M will have some utility as farnesyl transferase inhibitors, the more preferred compounds will exhibit an IC₅₀ of between 0.01.mu.M and 1.mu.M. The most preferred compounds will generally have an IC₅₀ of between about 0.01.mu.M and 0.3.mu.M.

### Patients

The present invention provides methods to determine the likelihood of therapeutic effectiveness of a farnesyl transferase inhibitor (FTI) in a patient, by determining whether the gene encoding the target farnesyl transferase beta subunit (FNTB) comprises at least one nucleic acid variance that causes an alteration in an amino acid residue, where the change in the amino acid residue is associated with resistance to a FTI. The absence of at least one variance in the target FNTB indicates that the FTI is likely to be effective. The patient's therapeutic regimen can then be designed to reflect the likely effectiveness of the FTI.

In one embodiment, the patient is affected with cancer, and the target FNTB for FTI therapy is encoded by the patient's own gene. In one preferred embodiment the cancer is a leukemia, including CML.

### Assays for Farnesyl Protein Transferases

In some embodiments of the invention, it is useful to assay farnesyl transferase activity in a composition. This is an important aspect of the invention in that such an assay system provides one with not only the ability to follow isolation and purification of the enzyme, but it also forms the basis for developing a screening assay for candidate inhibitors of the enzyme, discussed in more detail below.

As described below, one particularly preferred embodiment of the invention provides methods for identifying or screening for novel agents which inhibit the activity of the valiant FTases taught here, which are resistant to other FTIs.

The assay method generally includes simply determining the ability of a composition suspected of having farnesyl transferase activity to catalyze the transfer of farnesol to an acceptor protein or peptide. As noted above, a farnesyl acceptor protein or peptide is generally defined as a protein or peptide which will act as a substrate for farnesyl transferase and which includes a recognition site such as —C-A-A-X, as defined above.

Typically, the assay protocol is carried out using farnesyl pyrophosphate as the farnesol donor in the reaction. Thus, one will find particular benefit in constructing an assay wherein a label is present on the farnesyl moiety of farnesyl pyrophosphate, in that one can measure the appearance of such a label, for example, a radioactive label, in the farnesyl acceptor protein or peptide.

As with the characterization of the enzyme discussed above, the farnesyl acceptor sequence which are employed in connection with the assay can be generally defined by —C-A-A-X, with preferred embodiments including sequences such as —C—V—I-M—C—S—I-M, -I-C-A-I-M, etc., all of which have been found to serve as useful enzyme substrates. It is believed that most proteins or peptides that include a carboxy terminal sequence of —C-A-A-X can be successfully employed in farnesyl protein transferase assays. For use in the assay a preferred farnesyl acceptor protein or peptide will be simply a p21^{ras} protein. This is particularly true where one seeks to identify inhibitor substances, as discussed in more detail below, which function either as “false acceptors” in that they divert farnesylation away from natural substrates by acting as substrates in and or themselves, or as “pure” inhibitors which are not in themselves farnesylated. The advantage of employing a natural substrate such as p21.sup.ras is several fold, but includes the ability to separate the natural substrate from the false substrate to analyze the relative degrees of farnesylation.

However, for the purposes of simply assaying enzyme specific activity, e.g., assays which do not necessarily involve differential labeling or inhibition studies, one can readily employ short peptides as a farnesyl acceptor in such protocols, such as peptides from about 4 to about 10 amino acids in length which incorporate the recognition signal at their carboxy terminus. Exemplary farnesyl acceptor protein or peptides include but are not limited to CVIM; KKSKTKCVIM; TKCVIM; RASNRSCAIM; TQSPQNCSIM; CLIM; CVVM; and CVLS.

### Sequences of the Invention

The present invention provides a number of sequences which are useful for practicing the methods of the invention, including for nucleic acid detection. In one embodiment, the invention provides sequences and methods to detect specific alleles of farnesyl transferase beta, including detection of mutations associated with resistance to FTIs.

One embodiment provides the following two primers, which are useful for the amplification of human farnesyl transferase beta: FTBF 5′-ATG GCT TCT CCG AGT TCT TTC ACC-3′ (SEQ ID NO:1); and FTBR 5′-TCT CGA GTC CTC TAG TCG GTT GCA G-3′ (SEQ ID NO:2). Sequences of farnesyl transfe-rase genes are well known in the art, for example Genbank Accession Nos. L00635 and L10414 sequences of human farnesyl transferase beta (Andres, D. A., et al. “cDNA cloning of the two subunits of human CAAX farnesyltransferase and chromosomal mapping of FNTA and FNTB loci and related sequences”, Genomics 18 (1), 105-112 (1993)).

Another preferred embodiment of the invention provides primer sequences which are useful for the detection of specific alleles of human farnesyl transferase beta, as described in the following table:

| | | | | |
|---|---|---|---|---|
| Primer | | Amino | | SEQ |
| Name | Exon | Acid | SEQUENCE | ID NO: |

| | | | | |
|---|---|---|---|---|
| | | | GENOMIC DNA AMPLIFICATION PRIMERS | |
| INT_FTB_95F | 1 | 95R | 5′-TTT TCT CTC CTG TCT CTC TC-3′ | 3 |
| INT_FTB_95R | 1 | 95R | 5′-CTT GTC TCT CAG AGT TGA TG-3′ | 4 |
| INT_FTB_213F | 7 | 213L | 5′-TCA CTG AGC CTC ATT AGC TC-3′ | 5 |
| INT_FTB_213R | 7 | 213L | 5′TTC TGA AGT AGT GTC GTG AC-3′ | 6 |
| INT_FTB_242F | 8 | 242I | 5′-TTG TGT ACG TCC ACT CAC TG-3′ | 7 |
| INT_FTB_242R | 8 | 242I | 5′-AAG ACA GAG CAG CTG CTC AC-3′ | 8 |
| INT_FTB_305F | 9 | 305F | 5′-TGC TTC ACT CTG TGT CTA TG-3′ | 9 |
| INT_FTB_305R | 9 | 305F | 5′-ATC CAG GAT AGA CAG AGC TC-3′ | 10 |
| INT_FTB_361F | 11 | 361L | 5′-AGG GCT GGA GGA TGG GGC TTT TA-3′ | 11 |
| INT_FTB_361R | 11 | 361L | 5′-GCA TGG CTG CAG TGC TAT CAC GA-3′ | 12 |
| | | | Allele specific PCR primers | |
| AS_242I_R | 8 | 242I | 5′-ATG GGC TTC CAT CCC TGG TAT-3′ | 13 |
| AS_305_F | 9 | 305F | 5′-GCT GCT ACT CCT TCT GGC AGA-3′ | 14 |
| AS_361L_F | 11 | 361L | 5′-CCT GGC AAG TCG CGT GAT TTC TTA-3′ | 15 |
| | | | Site directed PCR primers | |
| FTB_C95R_F | 1 | 95R | 5′-GAT GCC TAT GAG CGT CTG GAT GCC AGC-3′ | 16 |
| FTB_C95R_R | 1 | 95R | 5′-GCT GGC ATC CAG ACG CTC ATA GGC ATC-3′ | 17 |
| FTB_W106R_F | | 106R | 5′-GGC TCT GCT ATA GGA TCC TGC AC-3′ | 18 |
| FTB_W106R_R | | 106R | 5′-GTG CAG GAT CCT ATA GCA GAG CC-3′ | 19 |
| FTB_P152S_F | | 152S | 5′-CCA CAC CTT GCA TCC ACA TAT GCA GCA-3′ | 20 |
| FTB_P152S_R | | 152S | 5′-TGC TGC ATA TGT GGA TGC AAG GTG TGG-3′ | 21 |
| FTB_A155T_F | | 155T | 5′-GCA CCC ACA TAT TCA GCA GTC AAT G-3′ | 22 |
| FTB_A155T_R | | 155T | 5′-CAT TGA CTG CTG AAT ATG TGG GTG C-3′ | 23 |
| FTB_P213L_F | 7 | 213L | 5′-CTC CGT AGC CTC GCC GAC CAA CAT CAT CAC-3′ | 24 |
| FTB_P213L_R | 7 | 213 | 5′-GTG ATG ATG TTG GTC GGC GAG GCT ACG GAG-3′ | 25 |
| FTB_G224S_F | | 224 | 5′-GAC CTC TTT GAG AGC ACT GCT GAA TGG-3′ | 26 |
| FTB_G224S_R | | 224 | 5′-CCA TTC AGC AGT GCT CTC AAA GAG GTC-3′ | 27 |
| FTB_G241E_F | | 241 | 5′-GGT GGC ATT GGC GAG GTA CCA GGG ATG-3′ | 28 |
| FTB_G241E_R | | 241 | 5′-CAT CCC TGG TAC CTC GCC AAT GCC ACC-3′ | 29 |
| FTB_242I_F | | 242 | 5′-GGC ATT GGC GGG ATA CCA GGG ATG GAA-3′ | 30 |
| FTB_242I_R | | 242 | 5′-TTC CAT CCC TGG TAT CCC GCC AAT GCC-3′ | 31 |
| FTB_E265K_F | | 265 | 5′-TAA TCC TCA AGA GGA AAC GTT CCT TGA AC-3′ | 32 |
| FTB_E265K_R | | 265 | 5′-GTT CAA GGA ACG TTT CCT CTT GAG GAT TA-3′ | 33 |
| FTB_M282V_F | | 282 | 5′-ACA AGC CGG CAG GTG CGA TTT GAA GGA-3′ | 34 |
| FTB_M282V_R | | 282 | 5′-TCC TTC AAA TCG CAC CTG CCG GCT TGT-3′ | 35 |
| FTB_E285K_F | | 285 | 5′-GCA GAT GCG ATT TAA AGG AGG ATT TCA GG-3′ | 36 |
| FTB_E285K_R | | 285 | 5′-CCT GAA ATC CTC CTT TAA ATC GCA TCT GC-3′ | 37 |
| FTB_A305T_F | | 305 | 5′-TCC TTC TGG CAG ACG GGG CTC CTG C-3′ | 38 |
| FTB_A305T_R | | 305 | 5′-GCA GGA GCC CCG TCT GCC AGA AGG A-3′ | 39 |
| FTB_F360S_F | | 360 | 5′-AAG TCG CGT GAT TCC TAC CAC ACC TGC-3′ | 40 |
| FTB_F361S_R | | 360 | 5′-GCA GGT GTG GTA GGA ATC ACG CGA CTT-3′ | 41 |
| FTB_Y361L_F | | 361 | 5′-TCG CGT GAT TTC TTA CAC ACC TGC TAC-3′ | 42 |
| FTB_Y361L_R | | 361 | 5′-GTA GCA GGT GTG TAA GAA ATC ACG CGA-3′ | 43 |
| FTB_Y361H_F | | 361 | 5′-TCG CGT GAT TTC CAC CAC ACC TGC TAC-3′ | 44 |
| FTB_Y361H_R | | 361 | 5′-GTA GCA GGT GTG GTG GAA ATC ACG CGA-3′ | 45 |
| FTB_Y361S_F | | 361 | 5′-CGC GTG ATT TCT CCC ACA CCT GCT AC-3′ | 46 |
| FTB_Y361S_R | | 361 | 5′-GTA GCA GGT GTG GGA GAA ATC ACG CG-3′ | 47 |

For allele specific PCR assays of cDNA, a PCR reaction can be performed using mixture of the three primers in each reaction: FTBF (SEQ ID NO:1), FTBR (SEQ ID NO:2), and one allele specific primer, for example AS_{—}242I_R (SEQ ID NO:13), AS_{—}305_F (SEQ ID NO:14), or AS_{—}361L_F (SEQ ID NO:15). For allele specific PCR assays of genomic DNA, a PCR reaction can be performed using a mix of three allele specific primer sets. For detection of a mutation at V242I, one uses INT_FTB_{—}242F (SEQ ID NO:7), INT_FTB_{—}242R (SEQ ID NO:8), and AS-242I_R (SEQ ID NO:13). For detection of a mutation at A305T, one uses INT_FTB_{—}305F (SEQ ID NO:9), INT_FTB_{—}305R (SEQ ID NO:10), and AS_{—}305SF (SEQ ID NO:14). For detection of a mutation at Y361L, one uses INT_FTB_{—}361F (SEQ ID NO:11), INT_FTB_{—}361R (SEQ ID NO:12), AS_{—}361L (SEQ ID NO:15). Each of these reaction is designed to give PCR amplification with the two wild-type primers (listed first). This reaction will occur in all samples mutant or not. In addition the AS primer (listed last) coupled to one of the wild-type primers will only amplify in the presence of mutation.

### DEFINITIONS

The terms “farnesyl protein transferase” and “FTase” and “farnesyl transferase” are used interchangeably herein. The terms “beta subunit of a farnesyl protein transferase” and “farnesyl protein transferase beta subunit” and “FNTB” are used interchangeably herein.

The term “farnesyl transferase activity decreasing nucleic acid variance” as used herein refers to a valiance (i.e. mutation) in the nucleotide sequence of a gene that results in a decreased activity. The decreased farnesyl transferase activity is a direct result of the variance in the nucleic acid and is associated with the protein for which the gene encodes.

The term “drug” or “compound” as used herein refers to a chemical entity or biological product, or combination of chemical entities or biological products, administered to a person to treat or prevent or control a disease or condition. The chemical entity or biological product is preferably, but not necessarily a low molecular weight compound, but may also be a larger compound, for example, an oligomer of nucleic acids, amino acids, or carbohydrates including without limitation proteins, oligonucleotides, ribozymes, DNAzymes, glycoproteins, siRNAs, lipoproteins, aptamers, and modifications and combinations thereof.

The term “genotype” in the context of this invention refers to the particular allelic form of a gene, which can be defined by the particular nucleotide(s) present in a nucleic acid sequence at a particular site(s).

The terms “variant form of a gene”, “form of a gene”, or “allele” refer to one specific form of a gene in a population, the specific form differing from other forms of the same gene in the sequence of at least one, and frequently more than one, variant sites within the sequence of the gene. The sequences at these variant sites that differ between different alleles of the gene are termed “gene sequence variances” or “variances” or “variants”. Other terms known in the art to be equivalent include mutation and polymorphism. In preferred aspects of this invention, the variances are selected from the group consisting of the variances listed in herein.

In the context of this invention, the term “probe” refers to a molecule which can detectably distinguish between target molecules differing in structure. Detection can be accomplished in a variety of different ways depending on the type of probe used and the type of target molecule. In certain embodiments, the probe can be detectably labeled. Thus, for example, detection may be based on discrimination of activity levels of the target molecule, but preferably is based on detection of specific binding. Examples of such specific binding include antibody binding and nucleic acid probe hybridization. Thus, for example, probes can include enzyme substrates, antibodies and antibody fragments, and preferably nucleic acid hybridization probes. In other embodiments, the probe itself is unlabeled, but it is used in a process where the product of the process can be detected; for example, in a PCR reaction.

As used herein, the terms “effective” and “effectiveness” includes both pharmacological effectiveness and physiological safety. Pharmacological effectiveness refers to the ability of the treatment to result in a desired biological effect in the patient. Physiological safety refers to the level of toxicity, or other adverse physiological effects at the cellular, organ and/or organism level (often referred to as side-effects) resulting from administration of the treatment. “Less effective” means that the treatment results in a therapeutically significant lower level of pharmacological effectiveness and/or a therapeutically greater level of adverse physiological effects.

The term “primer”, as used herein, refers to an oligonucleotide which is capable of acting as a point of initiation of polynucleotide synthesis along a complementary strand when placed under conditions in which synthesis of a primer extension product which is complementary to a polynucleotide is catalyzed. Such conditions include the presence of four different nucleotide triphosphates or nucleoside analogs and one or more agents for polymerization such as DNA polymerase and/or reverse transcriptase, in an appropriate buffer (“buffer” includes substituents which are cofactors, or which affect pH, ionic strength, etc.), and at a suitable temperature. A primer must be sufficiently long to prime the synthesis of extension products in the presence of an agent for polymerase. A typical primer contains at least about 5 nucleotides in length of a sequence substantially complementary to the target sequence, but somewhat longer primers are preferred. Usually primers contain about 15-26 nucleotides, but longer primers may also be employed.

A primer will always contain a sequence substantially complementary to the target-sequence, that is the specific sequence to be amplified, to which it can anneal. A primer may, optionally, also comprise a promoter sequence. The term “promoter sequence” defines a single strand of a nucleic acid sequence that is specifically recognized by an RNA polymerase that binds to a recognized sequence and initiates the process of transcription by which an RNA transcript is produced. In principle, any promoter sequence may be employed for which there is a known and available polymerase that is capable of recognizing the initiation sequence. Known and useful promoters are those that are recognized by certain bacteriophage polymerases, such as bacteriophage T3, T7 or SP6.

A “microarray” is a linear or two-dimensional array of preferably discrete regions, each having a defined area, formed on the surface of a solid support. The density of the discrete regions on a microarray is determined by the total numbers of target polynucleotides or polypeptides to be detected on the surface of a single solid phase support, preferably at least about 50/cm², more preferably at least about 100/cm², even more preferably at least about 500/cm², and still more preferably at least about 1,000/cm². As used herein, a DNA microarray is an array of oligonucleotide primers placed on a chip or other surfaces used to amplify or clone target polynucleotides. Since the position of each particular group of primers in the array is known, the identities of the target polynucleotides can be determined based on their binding to a particular position in the microarray.

The term “label” refers to a composition capable of producing a detectable signal indicative of the presence of the target polynucleotide in an assay sample. Suitable labels include radioisotopes, nucleotide chromophores, enzymes, substrates, fluorescent molecules, chemiluminescent moieties, magnetic particles, bioluminescent moieties, and the like. As such, a label is any composition detectable by spectroscopic, photochemical, biochemical, immunochemical, electrical, optical or chemical means.

The term “support” refers to conventional supports such as beads, particles, dipsticks, fibers, filters, membranes and silane or silicate supports such as glass slides.

The term “amplify” is used in the broad sense to mean creating an amplification product which may include, for example, additional target molecules, or target-like molecules or molecules complementary to the target molecule, which molecules are created by virtue of the presence of the target molecule in the sample. In the situation where the target is a nucleic acid, an amplification product can be made enzymatically with DNA or RNA polymerases or reverse transcriptases.

As used herein, a “biological sample” refers to a sample of tissue or fluid isolated from an individual, including but not limited to, for example, blood, plasma, serum, tumor biopsy, urine, stool, sputum, spinal fluid, pleural fluid, nipple aspirates, lymph fluid, the external sections of the skin, respiratory, intestinal, and genitourinary tracts, tears, saliva, milk, cells (including but not limited to blood cells), tumors, organs, and also samples of in, vitro cell culture constituent.

Preferably, the amino acid residue that is altered in the variant FNTB is one or more of the following residues: C95, W106, J107, P152, A155, V195, G196, L213, G224, G241, V242, E265, M282, E285, A305, F360, and Y361. In one preferred embodiment, the altered amino acid residue is one or more of the following residues: C95, W106, I107, P152, A155,G241, V242, aid Y361. In one embodiment, the altered amino acid residue is not P152. In one embodiment, the alerted amino acid residue is not Y361. In one embodiment, the alerted amino acid residue is not Y365. In one embodiment, the alerted amino acid residue is not R202. In one embodiment, the altered amino acid residue is one or more of the following mutations: C95R, W106R, I107V, P152S, A155S, V195D, G196R, L213P, G224S, G241E, V242I, E265K, M282V, E285K, A305T, F360S, Y361L, Y361S: and Y361H. In one preferred embodiment, the altered amino acid residue is one or more of the following mutations: C95R, W106R, 107V, P152S, A155S, G241E, V242I, Y361S, and Y361H. In one embodiment, the altered amino acid residue is not the mutation Y361L. In one embodiment, the altered amino acid residue is not the mutation Y361M. In one embodiment, the altered amino acid residue is not the mutation Y361I. In one embodiment, the altered amino acid residue is not the mutation Y361C. In one embodiment, the altered-amino, acid residue is not the mutation P152M.

Nucleic acid molecules can be isolated from a particular biological sample using any of a number of procedures, which are well-known in the art, the particular isolation procedure chosen being appropriate for the particular biological sample. For example, freeze-thaw and alkaline lysis procedures can be useful for obtaining nucleic acid molecules from solid materials; heat and alkaline lysis procedures can be useful for obtaining nucleic acid molecules from urine; and proteinase K extraction can be used to obtain nucleic acid from blood (Rolff, A et al. PCR: Clinical Diagnostics and Research, Springer (1994).

### Detection Methods

Determining the presence of a particular variance or plurality of variances in a farnesyl transferase gene, such as FNTB in a patient with or at risk for developing cancer, can be performed in a variety of ways. Such tests are commonly performed using DNA or RNA collected from biological samples, e.g., tissue biopsies, urine, stool, sputum, blood, cells, tissue scrapings, breast aspirates or other cellular materials, and can be performed by a variety of methods including, but not limited to, PCR, hybridization with allele-specific probes, enzymatic mutation detection, chemical cleavage of mismatches, mass spectrometry or DNA sequencing, including mini sequencing. In particular embodiments, hybridization with allele specific probes can be conducted in two formats: (1) allele specific oligonucleotides bound to a solid phase (glass, silicon, nylon membranes) and the labeled sample in solution, as in many DNA chip applications, or (2) bound sample (often cloned DNA or PCR amplified DNA) and labeled oligonucleotides in solution (either allele specific or short so as to allow sequencing by hybridization). Diagnostic tests may involve a panel of variances, often on a solid support, which enables the simultaneous determination of more than one variance. In an alternative embodiment, allele specific oligo nucleotides can be used to detect the present of a specific allele, including mutations, using PCR RFLP.

In one particularly preferred embodiment, the “PCR colony assay,” also known as the “polony assay,” can be used for the sensitive detection of nucleic acid variance(s). These methods are described in detail in Mitra et al., Proc. Nat'l. Acad. Sci. USA 100:5926-5931 (2003) and Nuc. Acids Res. 27: e34 (1999), which are hereby incorporated by reference in their entirety, and are also described below.

In another aspect, determining the presence of at least one decreasing nucleic acid variance in a farnesyl transferase gene such as FNTB may entail a haplotyping test. Methods of determining haplotypes are known to those of skill in the art, as for example, in WO 00/04194.

Preferably, the determination of the presence or absence of a farnesyl transferase activity decreasing nucleic acid variance involves determining the sequence of the variance site or sites by methods such as polymerase chain reaction (PCR). PCR RFLP is one preferred method for detecting specific alleles or mutations. In PCR RFLP, when the presence of a specific allele or mutation changes a restriction enzyme site, one can amplify the fragment of DNA including the specific allele and detect its presence by restriction enzyme digestion of the amplified PCR products. Alternatively, the determination of the presence or absence of a farnesyl-transferase activity decreasing. nucleic acid variance may encompass chain terminating DNA sequencing or minisequencing, oligonucleotide hybridization or mass spectrometry.

The methods of the present invention may be used to predict the likelihood of effectiveness of an farnesyl transferase targeting treatment in a patient in one embodiment, the patient is affected with or at risk for developing cancer. Preferably, cancers include but are not limited to leukemias, solid tumors, non-small lung cancers, and colorectal cancer. Leukemias, including chronic myelogenous leukemia (CML), are particularly preferred.

The present invention generally concerns the identification of variances in a gene encoding a farnesyl transferase which are indicative of the effectiveness of a farnesyl transferase targeting treatment in a patient with or at risk for developing cancer. Additionally, the identification of specific variances in the gene encoding the farnesyl transferase, in effect, can be used as a diagnostic or prognostic test. For example, the absence of at least one variance in the gene encoding the farnesyl transferase indicates that a patient will likely benefit from treatment with an farnesyl transferase targeting compound, such as, for example, a FTI.

Methods for diagnostic tests are well known in the art and disclosed in patent application WO 00/04194, incorporated herein by reference. In an exemplary method, the diagnostic test comprises amplifying a segment of DNA or RNA (generally after converting the RNA to cDNA) spanning one or more known variances in the sequence of the gene encoding the farnesyl transferase. This amplified segment is then sequenced and/or subjected to polyacrylamide gel electrophoresis in order to identify nucleic acid variances in the amplified segment.

### PCR

In one embodiment, the invention provides a method of screening for variants in the gene encoding the farnesyl transferase in a test biological sample by PCR. The method comprises the steps of designing degenerate primers for amplifying the target sequence, the primers corresponding to one or more conserved regions of the gene, amplifying reaction with the primers using, as a template, a DNA or cDNA obtained from a test biological sample and analyzing the PCR products. Comparison of the PCR products of the test biological sample to a control sample indicates variances in the test biological sample. The change can be either and absence or presence of a nucleic acid variance in the test biological sample.

Primers useful according to the present invention are designed using amino acid sequences of the protein or nucleic acid sequences of the FTase as a guide, e.g. SEQ ID NOs:3-47 for human farnesyl transferase beta.

For example the identical or highly, homologous, preferably at least 80%-85% more preferably at least 90-99% homologous amino acid sequence of at least about 6, preferably at least 8-10 consecutive amino acids. Most preferably, the amino acid sequence is 100% identical. Forward and reverse primers are designed based upon the maintenance of codon degeneracy and the representation of the various amino acids at a given position among the known gene family members. Degree of homology as referred to herein is based upon analysis of an amino acid sequence using a standard sequence comparison software, such as protein-BLAST using the default settings (Altschul et al., 1990, J. Mol. Biol. 215:403-410.http://www.ncbi.nlm.nih.gov/BLAST/)

Primers may be designed using a number of available computer programs, including, but not limited to Oligo Analyzer3.0; Oligo Calculator; NetPrimer; Methprimer; Primer3; WebPrimer; PrimerFinder; Primer9; Oligo2002; Pride or GenomePride; Oligos; and Codehop.

Primers may be labeled using labels known to one skilled in the art. Such labels include, but are not limited to radioactive, fluorescent, dye, and enzymatic labels.

Analysis of amplification products can be performed using any method capable of separating the amplification products according to their size, including automated and manual gel electrophoresis, mass spectrometry, and the like.

Alternatively, the amplification products can be separated using sequence differences, using SSCP, DGGE, TGGE, chemical cleavage or restriction fragment polymorphisms as well as hybridization to, for example, a nucleic acid arrays.

The methods of nucleic acid isolation, amplification and analysis are routine for one skilled in the art and examples of protocols can be found, for example, in the Molecular Cloning: A Laboratory Manual (3-Volume Set) Ed. Joseph Sambrook, David W. Russel, and Joe Sambrook, Cold Spring Harbor Laboratory; 3rd edition (Jan. 15, 2001), ISBN: 0879695773. Particularly useful protocol source for methods used in PCR amplification is PCR (Basics: From Background to Bench) by M. J. McPherson, S. G. Møller, R. Beynon, C. Howe, Springer Verlag; 1st edition (Oct. 15, 2000), ISBN: 0387916008.

### Solid Support and Probe

In an alternative embodiment, the detection of the presence or absence of the at least one nucleic-acid variance involves contacting a nucleic acid sequence corresponding to the desired region of the gene encoding the farnesyl transferase, identified above, with a probe. The probe is able to distinguish a particular form of the gene or the presence or a particular variance or variances, e.g., by differential binding or hybridization. Thus, exemplary probes include nucleic acid hybridization probes, peptide nucleic acid probes, nucleotide-containing probes which also contain at least one nucleotide analog, and antibodies, e.g., monoclonal antibodies, and other probes as discussed herein. Those skilled in the art are familiar with the preparation of probes with particular specificities. These skilled in the art will recognize that a variety of variables can be adjusted to optimize the discrimination between two variant forms of a gene, including changes in salt concentration, temperature, pH and addition of various compounds that affect the differential affinity of GC vs. AT base pairs, such as tetramethyl ammonium chloride. (See Current Protocols in Molecular Biology by F. M. Ausubel, R. Brent, R. E. Kingston, D. D. Moore, J. G. Seidman, K. Struhl and V. B. Chanda (Editors), John Wiley & Sons.)

Thus, in preferred embodiments, the detection of the presence or absence of the at least one variance involves contacting a nucleic acid sequence which includes at least one variance site with a probe, preferably a nucleic acid probe, where the probe preferentially hybridizes with a form of the nucleic acid sequence containing a complementary base at the variance site as compared to hybridization to a form of the nucleic acid sequence having a non-complementary base at the variance site, where the hybridization is carried out under selective hybridization conditions. Such a nucleic acid hybridization probe may span two or more variance sites. Unless otherwise specified, a nucleic acid probe can include one or more nucleic acid analogs, labels or other substituents or moieties so long as the base-pairing function is retained.

Such hybridization probes are well known in the art (see, e.g., Sambrook et al., Eds., (most recent edition), Molecular Cloning: A Laboratory Manual, (third edition, 2001), Vol. 1-3. Cold Spring Harbor Laboratory, Cold Spring Harbor, N.Y.). Stringent hybridization conditions will typically include salt concentrations of less than about 1 M, more usually less than about 500 mM and preferably less than about 200 mM. Hybridization temperatures can be as low as 5° C. but are typically greater than 22° C., more typically greater than about 30° C., and preferably in excess of about 37° C. Longer fragments may require higher hybridization temperatures for specific hybridization. Other factors may affect the stringency of hybridization, including base composition and length of the complementary strands, presence of organic solvents and extent of base mismatching; the combination of parameters used is more important than the absolute measure of any one alone. Other hybridization conditions which may be controlled include buffer type and concentration, solution pH, presence and concentration of blocking reagents (e.g., repeat sequences, Cot1 DNA, blocking protein solutions) to decrease background binding, detergent type(s) and concentrations, molecules such as polymers which increase the relative concentration of the polynucleotides, metal ion(s) and their concentration(s), chelator(s) and their concentrations, and other conditions known or discoverable in the art. Formulas may be used to predict the optimal melting temperature for a perfectly complementary sequence for a given probe, but true melting temperatures for a probe under a set of hybridization conditions must be determined empirically. Also, a probe may be tested against its exact complement to determine a precise melting temperature under a given set of condition as described in Sambrook et al, “Molecular Cloning,” 3^{rd} edition, Cold Spring Harbor Laboratory Press, 2001. Hybridization temperatures can be systematically altered for a given hybridization solution using a support associated with target polynucleotides until a temperature range is identified which permits detection of binding of a detectable probe at the level of stringency desired, either at high stringency where only target polynucleotides with a high degree of complementarity hybridize, or at lower stringency where additional target polynucleotides having regions of complementarity with tbe probe detectably hybridize above the background level provided from nonspecific binding to noncomplementary target polynucleotides and to the support. When hybridization is performed with potential target polynucleotides on a support under a given set of conditions, the support is then washed under increasing conditions of stringency (typically lowered salt concentration and/or increased temperature, but other conditions may be altered) until background binding is lowered to the point where distinct positive signals may be seen. This can be monitored in progress using a Geiger counter where the probe is radiolabeled, radiographically, using a fluorescent imager, or by other means of detecting probe binding. The support is not allowed to dry during such procedures, or the probe may become in irreversibly bound even to background locations. Where a probe produces undesirable background or false positives; blocking reagents are employed, or different regions of the probe or different probes are used until positive signals can be distinguished from background. Once conditions are found that provide satisfactory signal above background, the target polynucleotides providing a positive signal are isolated and further characterized. The isolated polynucleotides can be sequenced; the sequence can be compared to databank entries or known sequences where necessary, full-length clones can be obtained by techniques known in the art; and the polynucleotides can be expressed using suitable vectors and hosts to determine if the polynucleotide identified encodes a protein having similar activity to that from which the probe polynucleotide was derived.

### Solid Phase Support

The solid phase support of the present invention can be of any solid materials and structures suitable for supporting nucleotide hybridization and synthesis. Preferably, the solid phase support comprises at least one substantially rigid surface on which oligonucleotides or oligonucleotide primers can be immobilized. The solid phase support can be made of, for example, glass, synthetic polymer, plastic: hard non-mesh nylon or ceramic. Other suitable solid support materials are known and readily available to those of skill in the art. The size of the solid support can be any of the standard microarray sizes, useful for DNA microarray technology; and the size may be tailored to fit the particular machine being used to conduct a reaction of the invention. Methods and materials for derivatization of solid phase supports for the purpose of immobilizing oligonucleotides are known to those skill in the art and described in, for example, U.S. Pat. No. 5,919,523, the disclosure of which is incorporated herein by reference.

The solid support can be provided in or be part of a fluid containing vessel. For example, the solid support can be placed in a chamber with sides that create a seal along the edge of the solid support so as to contain the polymerase chain reaction (PCR) on the support. In a specific example the chamber can have walls on each side of a rectangular support to ensure that the PCR mixture remains on the support and also to make the entire surface useful for providing the primers.

The oligonucleotide or oligonucleotide primers of the invention are affixed, immobilized, provided, and/or applied to the surface of the solid support using any available means to fix, immobilize, provide and/or apply the oligonucleotides at a particular location on the solid support. For example, photolithography (Affymetrix, Santa Clara, Calif.) can be used to apply the oligonucleotide primers at particular position on a chip or solid support, as described in the U.S. Pat. Nos. 5,919,523, 5,837,832, 5,831,070, and 5,770,722, which are incorporated herein by reference. The oligonucleotide primers may also be applied to a solid support as described in Brown and Shalon, U.S. Pat. No. 5,807,522 (1998). Additionally, the primers may be applied to a solid support using a robotic system, such as one manufactured by Genetic MicroSystems (Woburn, Mass.), GeneMachines (San Carlos, Calif.) or Cartesian Technologies (Irvine, Calif.).

In one aspect of the invention, solid phase amplification of target polynucleotides from a biological sample is performed, wherein multiple groups of oligonucleotide primers are immobilized on a solid phase support. In a preferred embodiment, the primers within a group comprises at least a first set of primers that are identical in sequence and are complementary to a defined sequence of the target polynucleotide, capable of hybridizing to the target polynucleotide under appropriate conditions, and suitable as initial primers for nucleic acid synthesis (i.e., chain elongation or extension). Selected primers covering a particular region of the reference sequence are immobilized, as a group, onto a solid support at a discrete location. Preferably, the distance between groups is greater than the resolution of detection means to be used for detecting the amplified products. In a preferred embodiment, the primers are immobilized to form a microarray or chip that can be processed and analyzed via automated, processing. The immobilized primers are used for solid phase amplification of target polynucleotides under conditions suitable for a nucleic acid amplification means. In this manner, the presence or absence of a variety of potential variances in a gene encoding a farnesyl transferase can be determined in one assay.

A population of target polynucleotides isolated from a healthy individual can used as a control in determining whether a biological source has at least one farnesyl transferase activity decreasing variance in the gene encoding the farnesyl transferase. Alternatively, target polynucleotides isolated from healthy tissue of the same individual may be used as a control as above.

An in situ-type PCR reactions on the microarrays can be conducted essentially as described in e.g. Embretson et al., Nature 362:359-362 (1993); Gosden et al., BioTechniques 15(1):78-80 (1993); Heniford et al Nuc. Acid Res. 21(14):3159-3166 (1993); Long et al, Histochemistry 99:151-162 (1993); Nuovo et al, PCR Methods and Applications 2(4):305-312 (1993); Patterson et al Science 260:976-979 (1993).

Alternatively, variances in the gene encoding the farnesyl transferase can be determined by solid phase techniques without performing PCR on the support. A plurality of oligonucleotide probes, each containing a distinct variance in the gene encoding the farnesyl transferase in duplicate, triplicate or quadruplicate, may be bound to the solid phase support. The presence or absence of variances in the test biological sample may be detected by selective hybridization techniques, known to those of skill in the art and described above.

### Mass Spectrometry

In another embodiment, the presence or absence of farnesyl transferase activity decreasing nucleic acid variances in a gene encoding a farnesyl transferase are determined using mass spectrometry. To obtain an appropriate quantity of nucleic acid molecules on which to perform mass spectrometry, amplification may be necessary. Examples of appropriate amplification procedures for use in the invention include: cloning (Sambrook et al. Molecular Cloning: A Laboratory Manual, 3^{rd} Edition, Cold Spring Harbor Laboratory Press, 2001), polymerase chain reaction (PCR) (C. R. Newton and A. Graham, PCR, BIOS Publishers, 1994), ligase chain reaction (LCR) (Wiedmann, M., et al., (1994) PCR Methods Appl. Vol. 3, Pp. 57-64; F. Barnay Proc. Natl. Acad. Sci. USA 88, 189-93 (1991), strand displacement amplification (SDA) (G. Terrance Walker et al., Nucleic Acids Res. 22, 2670-77 (1994)) and variations such as RT-PCR (Higuchi, et al. Bio/Technology 11:1026-1030 (1993)), allele-specific amplification (ASA) and transcription based processes.

To facilitate mass spectrometric analysis, a nucleic acid molecule containing a nucleic acid sequence to be detected can be immobilized to a solid support. Examples of appropriate solid supports include beads (e.g. silica gel, controlled pore glass, magnetic, Sephadex/Sepharose, cellulose), flat surfaces or chips (e.g. glass fiber filters, glass surfaces, metal surface (steel, gold, silver, aluminum, copper and silicon), capillaries, plastic (e.g. polyethylene, polypropylene, polyamide, polyvinylidenedifluoride membranes or microtiter plates)); or pins or combs made from similar materials comprising beads or flat surfaces or beads placed into pits in flat surfaces such as wafers (e.g. silicon wafers).

Immobilization can be accomplished, for example, based on hybridization between a capture nucleic acid sequence, which has already been immobilized to the support and a complementary nucleic acid sequence, which is also contained within the nucleic acid molecule containing the nucleic acid sequence to be detected. So that hybridization between the complementary nucleic acid molecules is not hindered by the support, the capture nucleic acid can include a spacer region of at least about five nucleotides in length between the solid support and the capture nucleic acid sequence. The duplex formed will be cleaved under the influence of the laser pulse and desorption can be initiated. The solid support-bound base sequence can be presented through natural oligoribo- or oligodeoxyribonucleotide as well as analogs (e.g. thio-modified phosphodiester or phosphotriester backbone) or employing oligonucleotide mimetics such as PNA analogs (see e.g. Nielsen et al., Science, 254, 1497 (1991)) which render the base sequence less susceptible to enzymatic degradation and hence increases overall stability of the solid support-bound capture base sequence.

Prior to mass spectrometric analysis, it may be useful to “condition” nucleic acid molecules, for example to decrease the laser energy required for volatilization and/or to minimize fragmentation. Conditioning is preferably performed while a target detection site is immobilized. An example of conditioning is modification of the phosphodiester backbone of the nucleic acid molecule (e.g. cation exchange), which can be useful for eliminating peak broadening due to a heterogeneity in the cations bound per nucleotide unit. Contacting a nucleic acid molecule with an alkylating agent such as alkyliodide, iodoacetamide, β-iodoethanol, 2,3-epoxy-1-propanol, the monothio phosphodiester bonds of a nucleic acid molecule can be transformed into a phosphotriester bond. Likewise, phosphodiester bonds may be transformed to uncharged derivatives employing trialkylsilyl chlorides. Further conditioning involves incorporating nucleotides which reduce sensitivity for depurination (fragmentation during MS) such as N7- or N9-deazapurine nucleotides, or RNA building blocks or using oligonucleotide triesters or incorporating phosphorothioate functions which are alkylated or employing oligonucleotide mimetics such as PNA.

For certain applications, it may be useful to simultaneously detect more than one (mutated) loci on a particular captured nucleic acid fragment (on one spot of an array) or it may be useful to perform parallel processing by using oligonucleotide or oligonucleotide mimetic arrays on various solid supports. “Multiplexing” can be achieved by several different methodologies. For example, several mutations can be simultaneously detected on one target sequence by employing corresponding detector (probe) molecules (e.g. oligonucleotides or oligonucleotide mimetics). However, the molecular weight differences between the detector oligonucleotides D1, D2 and D3 must be large enough so that simultaneous detection (multiplexing) is possible. This can be achieved either by the sequence itself (composition or length) or by the introduction of mass-modifying functionalities M1-M3 into the detector oligonucleotide.

Preferred mass spectrometer formats for use in the invention are matrix assisted laser desorption ionization (MALDI), electrospray (ES), ion cyclotron resonance (ICR) and Fourier Transform. Methods of performing mass spectrometry are known to those of skill in the art and are further described in Methods of Enzymology, Vol. 193:“Mass Spectrometry” (J. A. McCloskey, editor), 1990, Academic Press, New York.

### Sequencing

In other preferred embodiments, determining the presence or absence of the at least one farnesyl transferase activity decreasing nucleic acid variance involves sequencing at least one nucleic acid sequence. The sequencing involves the sequencing of a portion or portions of the gene encoding the farnesyl transferase which includes at least one variance site, and may include a plurality of such sites. Preferably, the portion is 500 nucleotides or less in length, more preferably 100 nucleotides or less, and most preferably 45 nucleotides or less in length. Such sequencing can be carried out by various methods recognized by those skilled in the art, including use of dideoxy termination methods (e.g. using dye-labeled dideoxy nucleotides), minisequencing, and the use of mass spectrometric methods.

### Method of Treating a Patient

In one embodiment, the invention provides a method for selecting a treatment for a patient by determining the presence or absence of at least one farnesyl transferase activity decreasing nucleic acid variance in a gene encoding a farnesyl transferase. In another embodiment, the variance is a plurality of variances, whereby a plurality may include variances from one, two, three or more gene loci.

In certain embodiments, the absence of the at least one variance is indicative that the treatment will be effective or otherwise beneficial (or more likely to be beneficial) in the patient. Stating that the treatment will be effective means that the probability of beneficial therapeutic effect is greater than in a person at least one farnesyl transferase decreasing nucleic acid variance(s) in a gene encoding a farnesyl transferase.

The treatment will involve the administration of a farnesyl transferase inhibitor. The treatment may involve a combination of treatments, including, but not limited to a farnesyl transferase inhibitor in combination with other farnesyl transferase inhibitors, chemotherapy, radiation, etc. One preferred treatment provide co-administration of at least two farnesyl transferaseinhibitors.

Thus, in connection with the administration of a farnesyl transferase inhibitor, a drug which is “effective” in a patient indicates that administration in a clinically appropriate manner results in a beneficial effect for at least a statistically significant fraction of patients, such as a improvement of symptoms, a cure, a reduction in disease load, reduction in tumor mass or cell numbers, extension of life, improvement in quality of life, or other effect generally recognized as positive by medical doctors familiar with treating the particular type of disease or condition.

In a preferred embodiment, the farnesyl transferase inhibitor is lonafarnib (SCH66336), tipifarnib (R115777), L-778,123, or BMS21466. Other preferred FTIs are described above. Lonagarnib is a particularly preferred inhibitor.

### Kits

### PCR Kits

The present invention therefore also provides predictive, diagnostic, and prognostic kits comprising degenerate primers to amplify a target nucleic acid in a gene encoding a farnesyl transferase and instructions comprising amplification protocol and analysis of the results. The kit may alternatively also comprise buffers, enzymes, and containers for performing the amplification and analysis of the amplification products. The kit may also be a component of a screening, diagnostic or prognostic kit comprising other tools such as DNA microarrays. Preferably, the kit also provides one or more control templates, such as nucleic acids isolated from normal tissue sample, and/or a series of samples representing different variances in a gene encoding a farnesyl transferase.

In one embodiment, the kit provides two or more primer pairs, each pair capable of amplifying a different region of a gene encoding a farnesyl transferase and (each region a site of potential variance) thereby providing a kit for analysis of expression of several gene variances in a biological sample in one reaction or several parallel reactions.

Primers in the kits may be labeled, for example fluorescently labeled, to facilitate detection of the amplification products and consequent analysis of the nucleic acid variances. Primers in the kits may also be unlabeled, for example for methods in the nucleic acid variance is detected by the presence or absence of a specific restriction enzyme site, such as PCR RFLP.

In one embodiment, more than one variance can be detected in one analysis. A combination kit will therefore comprise of primers capable of amplifying different segments of a gene encoding a farnesyl transferase. The primers maybe differentially labeled, for example using different fluorescent labels, so as to differentiate between the variances.

The primers contained within the kit may include any of the primers taught above as SEQ ID NOs: 1-47. Primer pairs which are useful for detecting specific mutations are described herein, and include primers for amplification of genomic DNA (e.g. SEQ ID NOs. 3-12), primers for the detection of specific alleles (e.g. SEQ ID NOs: 13-15); and site directed PCR primers (e.g. SEQ ID NOs: 17-47).

### Solid Support

In another embodiment, the invention provides a kit for practicing the methods of the invention. In one embodiment, a kit for the detection of variances in a gene encoding a farnesyl transferase on a solid support is described. The kit can include, e.g. the materials and reagents for detecting a plurality of variances in one assay. The kit can include e.g. a solid support, oligonucleotide primers for a specific set of target polynucleotides, polymerase chain reaction reagents and components, e.g. enzymes for DNA synthesis, labeling materials, and other buffers and reagents for washing. The kit may also include instructions for use of the kit to amplify specific targets on a solid support. Where the kit contains a prepared solid support having a set of primers already fixed on the solid support, e.g. for amplifying a particular set of target polynucleotides, the design and construction of such a prepared solid support is described above. The kit also includes reagents necessary for conducting a PCR on a solid support, for example using an in situ-type or solid phase type PCR procedure where the support is capable of PCR amplification using an in situ-type PCR machine. The PCR reagents, included in the kit, include the usual PCR buffers, a thermostable polymerase (e.g. Taq DNA polymerase), nucleotides (e.g. dNTPs), and other components and labeling molecules (e.g. for direct or indirect labeling as described above). The kits can be assembled to support practice of the PCR amplification method using immobilized primers alone or, alternatively, together with solution phase primers.

Alternatively, the kit may include a solid support with affixed oligonucleotides specific to any number of farnesyl transferase variances, including but not limited to the following mutations in FNTB: C95R, W106R, 1107V, P152S, A155S, V195D, G196R, L213P, G224S, G241 E, V242I, E265K, M282V, E285K, A305T, F360S, Y361L, Y361S, and Y361H. Preferably, the mutations in FNTB are: C95R, W106R, 1107V, P152S, A 55S, G241E, V242I, Y361 S, and Y361H. In one embodiment, the mutation is not Y361L. In one embodiment, the mutation is not Y361M. In one embodiment, the mutation is not Y361I. In one embodiment, the mutation is not Y361C. In one embodiment, the mutation is not P152M. In one embodiment the mutation is not Y361S. In one embodiment, the mutation is not Y361H. A test biological sample may be applied to the solid support under selective hybridization conditions, for the determination of the presence or absence of variances in a gene encoding a farnesyl transferase

The methods of the present invention also encompass the identification of compounds that interfere with the farnesyl transferase activity of a variant form of a gene encoding a farnesyl transferase. The variant gene comprises at least one variance. Such compounds may, for example, be farnesyl transferase inhibitors. Methods for identifying compounds that interfere with farnesyl transferase activity are generally known to those of skill in the ail and are further described above. In general, compounds are identified, using the methods disclosed herein, that interfere with the farnesyl transferase activity characteristic of at least one variance in a gene encoding a farnesyl transferase. Such known variances are described above.

Once identified, such compounds are administered to patients in need of farnesyl transferase targeted treatment, for example, patients affected with or at risk for developing cancer.

The route of administration may be intravenous (I.V.), intramuscular (I.M.), subcutaneous (S.C.), intradermal (I.D.), intraperitoneal (I.P.), intrathecal (I.T.), intrapleural, intrauterine, rectal, vaginal, topical, intratumor and the like. The compounds of the invention can be administered parenterally by injection or by gradual infusion over time and can be delivered by peristaltic means.

### Example 1

### Methods

### Plasmids

The beta subunit of FTase was cloned into the ecoR1 sites of the pEYK3.1 retroviral vector²² generating pEYK-FTB for the random mutagenesis, and into the EcoR1 sites of the pBabe retroviral vector²³ generating pBABE-FTB for verification of resistance by de-novo mutation generation. K-Ras61L (a constitutively active form of K-Ras containing a substitution of glutamine to leucine at position 61) was cloned into the EcoR1 sites of the MSCV-IRES-GFP retroviral vector.

### Cell Lines

BaF3 cells are a murine 1L3 dependent cell line which can be made IL3 independent by the expression of certain oncogenes such as KRas-61L. We found that the BaF3-IKRas-61L cells grown in the absence of IL3 had increased sensitivity to lonafarnib as compared with BaF3 cells grown in the presence of IL3.

Random Mutagenesis

pEYK-FTB plasmid was used to transform XL-1 Red E. Coli according to manufacturer's directions (Stratagene). Cells were plated on zeocin agar plates and incubated at 37° C. for 24 to 30 hours. Bacterial colonies were then scraped off the plates and the mutated plasmid library isolated (previously described²⁴). 1 μg of the plasmid library was then used along with 1 μg of the pCL/Eco viral packaging construct²⁵ to transfect 10⁶ 293t cells for virus production. Media was changed at 24 hours and viral supernatant collected at 48 hours post transfection and used to infect 10⁶ BaF3-KRas-61L cells. Cells were plated 24 hours after infection in 6 well plates at a density of 5×10⁴ cells per well in 4 ml of soft agar media (54% RPMI, 16% DME, 10% inactivated FCS, and 20% agar solution:1.2% agar in PBS) in the presence of varying drug concentrations (diluent (DMSO), 1 μM, 5 μM, and 10 μM lonafarnib). Plated cells were incubated at 37° C. for 14 days. Individual drug resistant colonies were then picked and expanded in liquid media. Genomic DNA was then isolated followed by a PCR amplification of vector DNA using vector specific oligonucleotides (1785F 5′-CACCCCCACCGCCCTCAAAGTAG-3′ and Zeo52R 5′-TAGTTCCTCACCTTGTCGTATTAT-3′). This PCR product was then sequenced for the identification of mutations.

### Verification by Site Directed Mutagenesis

Mutations identified in the initial screen were recreated in the pBABE-FTB vector by site directed mutagenesis according to manufacturer's instructions (Stratagene—Quick change kit). 293t cells were transfected with pBabe-puro-FTB and pCL/Eco. BaF3 cells were infected with viral supernatant (as described above) and FTB expressing cells selected in puromycin. The ability of mutant FTB to confer drug resistance was assessed by two assays: a. a colony counting assay, where pBABE-FTB expressing cells were plated in soft agar (44% RPMI, 16% DME, 10% WeHi 3B conditioned media (a source of IL3), 10% inactivated FBS, 20% agar solution: 1.2% agar in PBS) in the presence of varying drug concentration. Cell colonies were counted after 14 days. b. western blot: 10⁶ cells were plated in RPMI, 10% IFS, 10% WeHi-3B conditioned media, at varying drug concentrations (diluent, 0.1 μM, 0.5 μM, 1 μM. 2.5 μM, 5 μM, 7 μM, and 10 μM). Cells were collected after 48 hours and lysed (using 150 mM NaCl, 20 mM Tris pH 7.4, 10 mM NaF, 1 mM EDTA, 1 mM ZnCl, 1 mM MgCl, 1% NP-40 (Sigma), 10% Glycerol). 50-70 μg total protein was separated by sulfate-polyacrylamide gel electrophoresis (SDS-PAGE) and transferred to a PVDF membrane. Membranes were then incubated with HDJ2 primary antibody (NeoMarkers).

Patients

Patients Were participants in a pilot study of SCH66336 and Gleevec (imatinib mesylate) in chronic myelogenous leukemia in MD Anderson, Tex. All patients signed an informed consent permitting the use of peripheral blood and bone marrow samples in this research. 10 ml peripheral blood and 2 ml bone marrow samples were collected from 10 patients and used to isolate RNA, and genomic DNA (using the RNeasy, and DNeasy kits respectively, Qiagen, Hilden, Germany). cDNA was made from 1 μg RNA using the First Strand kit (Roche, Basel, Switzerland).

### Patient FTase β Sequencing

Patient FTase β was PCR amplified either from RNA, or from genomic DNA, cloned into the TOPO cloning vector according to manufacturers' instructions (TOPO cloning technology, Invitrogen, Carlsbad, Calif.) and used to transform E. Coli. Individual E. Coli colonies, each harboring a single PCR amplicon, were then isolated and sequenced.

### Competitive Proliferation Assay

1000 mutant cells and 1000 wild-type cells per 1 ml of RPMI media (10% inactivated FBS, 10% WeHi-3B conditioned media) were plated in each of a 24 well plate and allowed to proliferate for 10 days. Cell suspensions were mixed and allowed to proliferate for 5 days. 75% of the cells were then removed, replaced by fresh media and allowed to proliferate for an additional 3 days. Genomic DNA was then extracted (DNeasy kit, Qiagen, Hilden, Germany) and used as template for a PCR reaction using the following pBabe vector specific PCR primers: pBabeF-5′-CTTTATCCAGCCCTCAC-3′, pBabeR-5′-ACCCTAACTGACACACATTCC-3′ and sequenced (by direct sequencing). Sequence chromatograms were analyzed for the relative contribution of wild-type versus mutant cells. Results were verified by 2 independent experiments.

### Cell Proliferation Comparison

1000 mutant (Y361L or C95R) or wild-type cells were plated in RPM1 (10% inactivated FBS, 10% WeHi-3B conditioned media) and allowed to proliferate for 10 days. Cells were counted using trypan blue exclusion every 48 hours and doubling times charted.

### Results

### In-Vitro Mutagenesis Screen for the Identification of FTase β Mutations Causing Lonafarnib Resistance

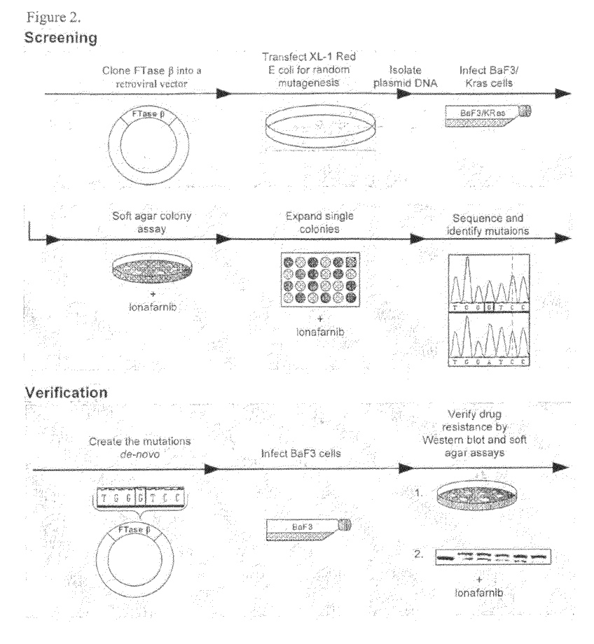

In identify mutations in FTase causing lonafarnib resistance we have adapted a methodology we have previously developed for the identification of BCR/ABL mutations causing imatinib resistance^{24,26} (see FIG. 2 for strategy outline). Briefly. we cloned the beta subunit of FTase (FNTB) into the pEYK3.1 retroviral vector generating pEYK-FTB. We next transfected XL1-Red E. coli cells (Stratagene, La Jolla, Calif.) that are deficient in DNA collection mechanisms thus generating a library of randomly mutated pEYK-FTB. This library was introduced, by retroviral infection, into BaF3 cells expressing K-Ras61L, which were found to be highly sensitive to lonafarnib (data not shown). BaF3 is a murine pro-B cell line which depends on the presence of IL3 for survival. The introduction of K-Ras-61L, a constitutively active form of K-Ras, allows the cells to survive in the absence of IL3. BaF3/K-Ras-61L cells infected with the pEYK-FTB mutagenized library were grown in soft agar in the presence of varying concentrations of lonafarnib (1 μM and 5 μM). Resistant cell colonies were picked and expanded. Genomic DNA was isolated from the cell expansions, and FNTB was sequenced yielding the following 17 mutations: C95R, W106R, P152S, A155S, V195D, G196R, G224S, G241E, V242I, E265K, M282V, E285K, A305T, F360S, Y361H, Y361L Y361S. 14 of these mutations are located on the surface of the active site of FTase (C95R, W106R, P152S, A155S, G241E, V242I, E265K, M282V, E285K, A305T, F360S, Y361H, Y361L, Y361S).

### Verification of Random Mutagenesis Screen Results

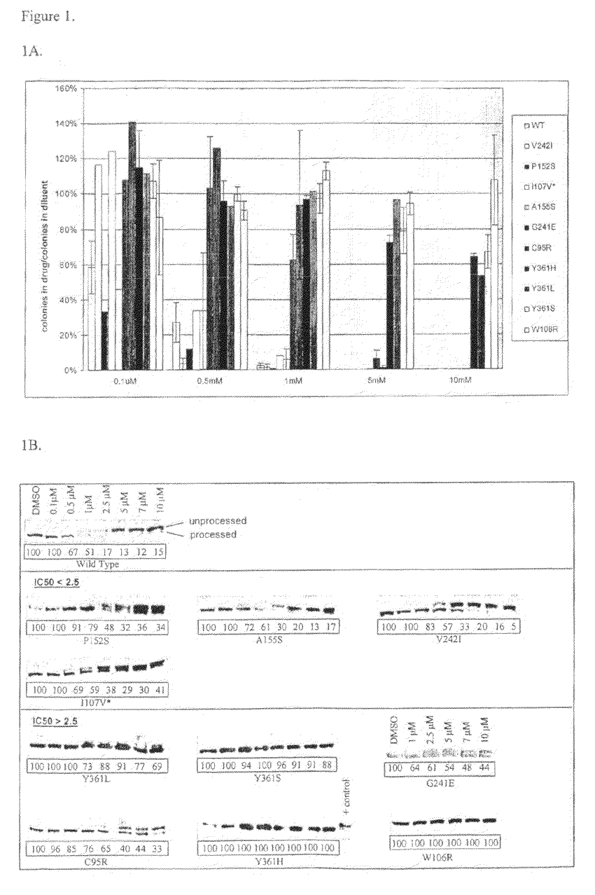

To verify the ability of the mutations identified in the random mutagenesis screen to confer lonafarnib resistance, wild-type FNTB was cloned into the pBabe-puro retroviral vector—generating pB-FTB. Each of the mutations identified in the screen was recreated de-novo in pB-FTB by site-directed mutagenesis (QuickChange kit, Stratagene. La Jolla, Calif.). BaF3 cells (without K-Ras61L expression) were infected with mutant pB-FTB and grown in the presence of lonafarnib. Lonafarnib resistance of each mutant was verified by two assays. The first is a soft agar plating assay where cells were plated in the presence of varying lonafarnib concentrations and allowed to proliferate for 14 days. Drug resistance was measured as a ratio between the number of colonies formed in drug to the number of colonies formed in diluent alone (see FIG. 1A). The second assay is a western blot analysis of mutation harboring cells grown in varying drug concentrations. Protein) farnesylation can be visualized by western blot since farnesylated proteins have a faster migration on a gel than unfarnesylated ones, due to the post farnesylation truncation of 3 C-terminal amino acids. In this assay, we assessed the proportion of farnesylated HDJ2 protein under varying drug concentrations. HDJ2 is a farnesylated chaperone protein used here as a convenient bio-marker (FIG. 1B). Of the 17 mutations originally identified 9 showed drug resistance by western blot and 7 by soft agar proliferation assay (C95R, W106R, A155S, G241E, Y361L, Y361H, Y361S). All 9 verified mutations were located at the active site of FTase, and 5 of them were found to be in direct contact of lonafarnib. An additional mutation (I107V) identified in a patient was subsequently tested and verified as well, bringing the number of verified resistance conferring mutations to 10 (see discussion).

### Patient Studies

Based on the ability of FTase β mutations to confer lonafarnib resistance in cell culture, we hypothesized that we may find the same mutations emerging in lonafarnib treated patients. For that reason we collected blood and bone marrow samples from patients enrolled in a clinical study at MD Anderson, Tex., of a combinatorial treatment of lonafarnib+imatinib (see table 2). All patients entering the study were imatinib refractory, with the rationale that the addition of lonafarnib will re-sensitize imatinib response. Samples were collected at baseline, and every 3 months thereafter. We searched for mutation in FNTB in patient samples by PCR amplification of FNTB from cDNA and from exons 4 and 11 of genomic DNA (where the majority of the mutations defined by our screen could be found) and sequenced between 20 and 50 separate PCR amplicons for each patient (as described in Methods). We have identified a number of mutations previously seen in our in-vitro screen, both in baseline samples and in samples taken after initiation of treatment.

TABLE 2

| | | | | | BCR/ | |
|---|---|---|---|---|---|---|
| | | Response | Response | BCR/ABL | ABL | FTase |
| Patient | Disease | to | duration | positivity | muta- | muta- |
| no. | stage | therapy | (months) | (%) | tions | tions |
| 1 | AP | Partial HR | 10 | 100 | E292V | I107V |
| 2 | AP | None | 0 | 100 | G250E, | Y361L |
| | | | | | M351T | C95R |
| 3 | CP | Partial CR | <15 | | None | None |
| 4 | CP | HR | 12 | | None | None |
| 5 | | None | 0 | | | None |
| 6 | | None | 0 | | | None |
| 7 | CP | None | 0 | 100 | G250E | None |
| 8 | | CHR | | | F359V | None |
| 9 | | Stable | | | E279L | None |
| | | disease | | | | |

AP—Accelerated phase.
CP—Chronic phase.
HR—Hematologic response.
CHR—Complete hematologic response.
SD—stable disease.

Patient 1 bad a V107I mutation found twice independently in a sample taken 3 months after treatment initiation. This mutation was not identified in our screen, however mutation in the adjacent residue W106R was found to confer strong resistance. We have recreated V107I de-novo, and verified its activity both in soft agar and by western blot (see FIG. 1).

We found two mutations Y361L, and C95R in patient 2 at a sample taken 3 months after treatment initiation.

A Y361H mutation was also found in a single baseline sample from an additional patient. No further samples were collected due to the patient's decision to withdraw from the study.

### Y361 Mutations Confer Growth Advantage in the Absence of Lonafarnib

The presence of a Y361H mutation in a patient sample taken before the initiation of lonafarnib treatment prompted us to assess the effect of mutations on cell proliferation in the absence of drug: For that purpose BaF3 cells expressing a mutant Fase β allele were equally mixed with cells expressing wild-type FTase β, plated, and allowed to proliferate for 8 days (see methods). Cells were then collected and FTase β sequenced. All Y361L, Y361H, or Y361S/wild-type expressing cell mixtures tested had a clear predominance of the mutant expressing cells in 2 independent experiments.

### Discussion

Lonafarnib is a highly specific small molecule inhibitor of FTase which is currently being evaluated in clinical trials (for various leukemias, breast cancer, and other cancers) both as monotherapy and in combination with other agents. We have reasoned that the highly specific nature of this inhibitor, is likely to render it susceptible to escape mutations that will prevent drug binding while still maintaining FTase's enzymatic activity. Mutations causing drug resistance have been well documented for the BCR/ABL inhibitor imatinib, and recently also for inhibitors of other protein kinases such as gefitinib, erlotinib, and PKC412¹⁹⁻²¹. Here we aim to demonstrate that drug resistance due to mutations in the target protein can cause lonafarnib resistance and is thus, not restricted to protein kinases. In addition to our findings in a cell culture model, we also find that the development of lonafarnib resistance due to FTase mutations has clinical relevance since we were able to detect such mutations in lonafarnib treated patients.

We have performed an in-vitro mutagenesis of the β subunit of FTase using a library of randomly mutated FNTB. This library was used to infect KRas61L expressing BaF3 cells grown in the absence of IL3. The KRas-BaF3 cells were chosen for the random mutagenesis because of their high sensitivity to lonafarnib. In subsequent verification experiments we have switched to BaF3 cells grown in the presence of IL3 in order to further distinguish mutants rendering robust drug resistance, likely to have clinical relevance. 17 mutations were isolated in the initial random mutagenesis experiment. Each of these mutations was then generated de-novo in a retroviral vector used to infect BaF3 cells. Mutation harboring cells were tested for their lonafarnib resistance by two assays. A soft agar assay, which measures the ability of cells to proliferate in the presence of drug (upon which the cellular IC50 is defined), and a western blot analysis of HDJ2 farnesylation, which is a measure of FTase activity (upon which the molecular IC50 is defined, see Table 1 mid FIG. 1). Once mutations were verified, we modeled them onto the FTase-lonafarnib co-crystal²⁷.

TABLE 1

| | Cellular | Molecular | Smallest distance |
|---|---|---|---|
| Mutation | IC50 (μM) | IC50 (μM) | from Lonafarnib (Å) |
| Wild-type | 0.09 | 0.8 | — |
| C95R | 1.8 | | 3.1 |
| W106R | >10 | >10 | 3.4 |
| P152S | 0.3 | 1.4 | 7.3 |
| A155S | 0.4 | 1.05 | 7.6 |
| G241E | 0.6 | | 11.8 |
| V242I | | 1.3 | 13.5 |
| Y361H | | | 3.1 |
| Y361L | 9.9 | | 3.1 |
| Y361S | | | 3.1 |

9 of the 17 mutations were verified to confer lonafarnib resistance to varying degrees. While all 9 mutants show lonafarnib resistance by western blot, 3 of them, P152S, A155S, and V242I conferred relatively weak resistance with molecular IC50 less then 2.5 μM. Cells harboring the other 6 mutations (G241E, C95R, Y361L, Y361S, Y361H, and W106R) had a molecular IC50>2.5, and also retained the ability to form colonies in the presence of lonafarnib in soft agar (FIG. 1). Of interest is the W106R mutation which confers the highest drug resistance of all mutants found. The growth of cells harboring this mutation in drug was comparable to cells grown in diluent, and the western blot analysis of HDJ2 shows full farnesylation (100%) in all drug concentrations. Modeling of this mutation on to the co-crystal structure of lonafarnib and FTase reveals a close contact between the tryptophan residue and lonafarnib along the length of the amino acid side chain. A substitution of this amino acid to an arginine, thus, is expected to disrupt van der waals interactions critical for lonafarnib's binding to the FTase β subunit. Similarly the three Y361 substitutions (to leucine, serine, and histidine) show strong resistance in both the soft agar and western blot analyses. This amino acid, as well, comes into close contact with lonafarnib along its side chain explaining the critical role it plays for lonafarnib binding. C95 comes into contact with lonafarnib only at the tip of its side chain. Drug resistance caused by the substitution to arginine, can be completely overcome by increasing the drug concentration to 5 μM. The other 4 mutations at residues P152, A155, G241, and V242 do not come into direct contact with the drug. Therefore, their effect on drug resistance may be a result of conformational changes to the active site. We-find that they cause a mild drug resistance that may still play a clinical role with trough plasma concentrations reported to reach only 1.5 μM⁸. Interestingly, mutations in the amino acid yeast homologues of 152 and 361 were previously reported to alter FTase substrate specificity (in yeast: amino acid 159 and 362 respectively). Such mutants had increased ability to farnesylate substrates terminating with a Leucine, which are typically prenylated by another prenyltransferase-geranylgeranyl transferase I (GGTase I)²⁸. Therefore, these mutations may result in increased farnesylation efficiency of some substrates.

To assess the clinical implications that mutations in FTase may have for patients treated with lonafarnib we collected blood samples from CML patients participating in a clinical trial using a lonafarnib and imatinib combination treatment. All patients recruited were imatinib refractory. Blood samples were collected at baseline and every 3 months thereafter. We speculated that treatment with lonafarnib may give a growth advantage to leukemic cells harboring FTase β mutations conferring drug resistance. For this purpose we sequenced FTase β both from RNA and genomic DNA isolated from patient blood samples. To increase the sensitivity of mutation detection we cloned the PCR amplicons into an expression vector (TOPO cloning technology, Invitrogen, Carlsbad, Calif.) and individually sequenced between 10-50 clones for each patient. Once we have detected a mutation previously identified in our in-vitro screen we verified its existence by allele specific PCR and/or by a second FTase β sequencing from an independent cDNA and PCR reaction. We found mutations of interest in three patients.

Patient 1 had no mutations that were previously identified in our screen, however, this patient did have a V107I mutation which we sequenced twice from 2 independent cDNA and PCR reactions. Since a mutation in the adjacent residue (W106R) was found in our screen to cause high resistance to lonafarnib we generated V107I de-novo and verified its ability to confer lonafarnib resistance (see FIG. 1).

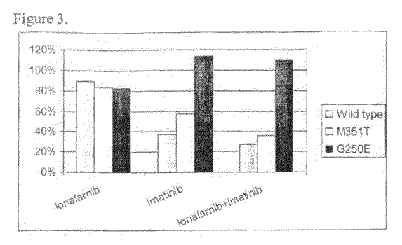

Patient 2 had 2 FTase P mutations previously identified in the in-vitro screen, C95R and Y361L. Both of these mutations were detected at samples acquired 3 months past treatment initiation. However, we were unable to detect the presence of either of these mutations by cloning and sequencing of samples taken at 6 months past treatment initiation. This patient, who was taken off the study after 6 months due to lack of clinical response, entered the study with 2 BCR/ABL mutations G250E and M351T, both conferring resistance to imatinib. The lonafarnib+imatinib drug combination was previously reported to be completely ineffective against another BCR/ABL mutant T351I¹². We tested the effect of the drug combination on BaF3 cells harboring G250E and M351T and found that M351T was partially resistant and G250E fully resistant to the combination treatment (FIG. 3). It may, therefore be, that the presence of a strong BCR/ABL mutation such as the G250E seen in patient 2 is responsible for the lack of response to treatment.

The fact that mutations seen in patient 2 appear at baseline, suggest that the presence of these mutations may confer growth advantage even in the absence of lonafarnib. To investigate this possibility we preformed a competitive proliferation assay of a few of the FTase β mutants identified in our screen. We plated equal numbers of wild-type FTase and mutant FTase in a 24 well dish and allowed them to proliferate for 8 days. We then isolated DNA from each of these wells and sequenced FTase β. The C95R/WT mixed wells were all predominantly wild-type suggesting that C95R does not confer a growth advantage. In contrast, all wells containing the Y361/WT cell mixtures (both Y361H/WT and Y361S/WT) showed predominance of the Y361 mutant form exclusively. This suggests that a substitution in residue 361 into either H or S confers a growth advantage.

The references cited below and throughout the application are incorporated herein by reference in their entirety.

### REFERENCES

1. Deininger M, Buchdunger E, Druker B J. The development of imatinib as a therapeutic agent for chronic myeloid leukemia. Blood. 2005; 105:2640-2653.
2. Hampton T. Looking Beyond Imatinib Next Line of Targeted Drugs for CML Shows Promise. JAMA. 2006; 295:370.
3. Kohl N E, Mosser S D, deSolms S J, et al., Selective inhibition of ras-dependent transformation by a farnesyltransferase inhibitor. Science. 1993; 260:1934-1937.
4. Castro A F, Rebhun J F, Clark G J, Quilliam L A. Rheb binds tuberous sclerosis complex 2 (TSC2) and promotes S6 kinase activation in a rapamycin- and farnesylation-dependent manner. J Biol Chem. 2003; 278:32493-32496.
5. Ashar H R James L, Gray K, et al., Farnesyl transferase inhibitors block the farnesylation of CENP-E and CENP-F and alter the association of CENP-E with the microtubules. J Biol Chem. 2000; 275:30451-30457.
6. Karp J E, Lancet J E, Kaufmann S H, et al., Clinical and biologic activity of the farnesyltransferase inhibitor R115777 in adults with refractory and relapsed acute leukemias: a phase 1 clinical-laboratory correlative trial. Blood. 2001; 97:3361-3369.
7. Adjei A A, Erlichman C, Davis J N, et al., A Phase I trial of the farnesyl transferase inhibitor SCH66336: evidence for biological and clinical activity. Cancer Res. 2000; 60:1871-1877.
8. Eskens F A, Awada A. Cutler D L, et al., Phase 1 and pharmacokinetic study of the oral farnesyl transferase inhibitor SCH 66336 given twice daily to patients with advanced solid tumors. J Clin Oncol. 2001; 19:1167-1175.
9. David E, Sun S Y, Waller E K, Chen J, Khuri F R, Lonial S. The combination of the farnesyl transferase inhibitor lonafarnib and the proteasome inhibitor bortezomib induces synergistic apoptosis in human myeloma cells that is associated with down-regulation of p-AKT. Blood. 2005; 106:4322-4329.
10. Jorgensen H G, Allan E K, Graham S M, et al., Lonafarnib reduces the resistance of primitive quiescent CML cells to imatinib mesylate in vitro. Leukemia. 2005; 19:1184-1191.
11. Adjei A A, Davis J N, Bruzek L M, Erlichman C, Kaufmann S H. Synergy of the protein farnesyltransferase inhibitor SCH66336 and cisplatin in human cancer cell lines. Clin Cancer Res. 2001; 7:1438-1445.
12. Hoover R R, Mahon F X, Melo V, Daley G Q. Overcoming ST1571 resistance with the farnesyl transferase inhibitor SCH66336. Blood. 2002; 100:1068-1071.
13. Basso A D, Kirschmeier P, Bishop W R. Thematic review series: Lipid Posttranslational Modifications. Farnesyl transferase inhibitors. J Lipid Res. 2006; 47:15-31.
14. Jabbour E, Kantarjian H, Cortes J. Clinical activity of farnesyl transferase inhibitors in hematologic malignancies: possible mechanisms of action. Leuk Lymphoma. 2004; 45:2187-2195.
15. Buckner F S, Eastman R T, Yokoyama K, Gelb M H, Van Voorhis W C. Protein farnesyl transferase inhibitors for the treatment of malaria and African trypanosomiasis. Curr Opin Investig Drugs. 2005; 6:791-797.
16. Mallampalli M P, Huyer G, Bendale P, Gelb M H; Michaelis S. Inhibiting farnesylation reverses the nuclear morphology defect in a HeLa cell model for Hutchinson-Gilford progeria syndrome. Proc Natl Acad Sci USA. 2005; 102:14416-14421.
17. Capell B C, Erdos M R, Madigan J P, et al., Inhibiting farnesylation of progerin prevents the characteristic nuclear blebbing of Hutchinson-Gilford progeria syndrome. Proc Natl Acad Sci U S A. 2005; 102:12879-12884.
18. Toth J I, Yang S H, Qiao X, et al., Blocking protein farnesyltransferase improves nuclear shape in fibroblasts from humans with progeroid syndromes. Proc Natl Acad Sci USA. 2005; 102:12873-12878.
19. Heidel F, Solem F1(, Breitenbuecher F. et al., Clinical resistance to the kinase inhibitor PKC412 in acute myeloid leukemia by mutation of Asn-676 in the FLT3 tyrosine kinase domain. Blood. 2006; 107:293-300.
20. Kobayashi S, Boggon T J, Dayaram T, et al., EGFR mutation and resistance of non-small-cell lung cancer to gefitinib. N Engl J. Med. 2005; 352:786-792.
21. Pao W, Miller V A, Politi K A, et al., Acquired resistance of lung adenocarcinomas to gefitinib or erlotinib is associated with a second mutation in the EGFR kinase domain. PLoS Med. 2005; 2:e73.
22. Koh E Y, Clien T, Daley G Q. Novel retroviral vectors to facilitate expression screens in mammalian cells. Nucleic Acids Res. 2002; 30:e142.
23. Morgenstern J P, Land H. Advanced mammalian gene transfer: high titre retroviral vectors with multiple drug selection markers and a complementary helper-free packaging cell line. Nucleic Acids Res. 1990; 18:3587-3596.
24. Azam M, Raz T, Nardi V, Opitz S L, Daley G Q. A screen to identify drug resistant variants to target-directed anti-cancer agents. Biol Proced Online. 2003; 5:204-210.
25. Naviaux R K, Costanzi E, Haas M, Verma I M. The pCL vector system: rapid production of helper-free, high-titer, recombinant retroviruses. J. Virol. 1996; 70:5701-5705.
26. Azam M. Latek R R, Daley G Q. Mechanisms of autoinhibition and STI-571/imatinib resistance revealed by mutagenesis of BCR-ABL. Cell. 2003; 112:831-843.
27. Strickland C L, Weber P C, Windsor W T, et al. Tricyclic farnesyl protein transferase inhibitors: crystallographic and calorimetric studies of structure-activity relationships. J Med Chem. 1999; 42:2125-2135.
28. Del Villar K, Mitsuzawa H, Yang W, Sattler I, Tamanoi F. Amino acid substitutions that convert the protein substrate specificity of farnesyltransferase to that of geranylgeranyltransferase type I. J Biol. Chem. 1997; 272:680-687.
29. Del Villar K, Urano J, Guo L, Tamnanoi F: A mutant form of human protein farnesyltransferase exhibits increased resistance to famnesyltransferase inhibitors. J Biol Chem 274:27010-7, 1999

1. A method for determining the likelihood of effectiveness of a farnesyl transferase inhibitor in a patient comprising: determining whether the gene encoding the farnesyl transferase beta subunit (FNTB) in the biological sample obtained from the patient comprises at least one nucleic acid variance that causes a change in an amino acid residue, wherein the change in the amino acid residue is associated with resistance to a farnesyl transferase inhibitor, and wherein the absence of the least one nucleic acid variance indicates that the farnesyl transferase inhibitor is likely to be effective in said patient.

2. The method of claim 1, wherein said patient has or is suspected of having cancer.

3. The method of claim 1, wherein the presence or absence of a nucleic acid variance in the FNTB gene is determined before the administration of a pharmaceutical composition comprising a farnesyl tranferase inhibitor to the patient.

4. The method of claim 1, wherein the presence or absence of a nucleic acid variance in the FNTB gene is determined after administration of a pharmaceutical composition comprising a farnesyl transferase inhibitor to the patient has commenced.

5. The method of claim 1, wherein the farnesyl transferase inhibitor is selected from the group consisting of lonafarnib (SCH66336), tipifarnib (R115777), L-778,123, and BMS21466.

6. The method of claim 5, wherein the farnesyl transferase inhibitor is lonafarnib.

7. The method of claim 1, wherein the nucleic acid variance is an in frame deletion or substitution.

8. The method of claim 1, wherein the nucleic acid variance decreases farnesyl transferase activity.

9. The method of claim 1, wherein the nucleic acid variance changes an amino acid within the active site of the farnesyl transferase enzyme.

10. The method of claim 1, wherein the nucleic acid variance changes an amino acid residue in the corresponding protein, wherein the amino acid residue is selected from the group consisting of C95, W106, I107, P152, A155, G241, V242, Y361, and Y361.

11. The method of claim 10, wherein the nucleic acid variance changes an amino acid residue in the corresponding protein, wherein the amino acid residue is selected from the group consisting of C95R, W106R, I107V, P152S, A155S, G241E, V242I, Y361S, and Y361H.

12. The method of claim 1, wherein the altered amino acid residue is not Y361.

13. The method of claim 12, wherein the altered amino acid residue is not Y361L.

14. The method of claim 12, wherein the altered amino acid residue is not Y361C.

15. A method for determining the resistance of a cell to a farnesyl transferase inhibitor, comprising:
(a) providing a test cell(s); and
(b) determining the presence or absence of at least one nucleic acid variance in a gene encoding the farnesyl transferase beta subunit (FNTB) in the test cell(s), wherein the presence of the at least one nucleic acid variance indicates that the farnesyl transferase inhibitor is likely to be less effective in said test cells.

16. The method of claim 15, wherein the test cell(s) is obtained from a biological sample obtained from an individual.

17. The method of claim 16, wherein the individual has or is suspected to have cancer, and the gene is the individual's FNTB gene.

18. The method of claim 15, wherein the farnesyl transferase inhibitor is selected from the group consisting of lonafarnib (SCH66336), tipifarnib (R115777), L-778,123, and BMS214662.

19. The method of claim 18, wherein the farnesyl transferase inhibitor is lonafarnib.

20. The method of claim 15, wherein the nucleic acid variance decreases farnesyl transferase activity.

21. The method of claim 15, wherein the nucleic acid variance changes an amino acid within the active site of the farnesyl transferase enzyme.

22. The method of claim 15, wherein the nucleic acid variance is a deletion, substitution, or insertion.

23. The method of claim 15, wherein the nucleic acid variance changes an amino acid residue in the corresponding protein, wherein the amino acid residue is selected from the group consisting of C95, W106, I107, P152, A155, G241, V242, Y361, and Y361.

24. The method of claim 23, wherein the nucleic acid variance changes an amino acid residue in the corresponding protein, wherein the amino acid residue is selected from the group consisting of C95R, W106R, I107V, P152S, A155S, G241E, V242I, Y361S, and Y361H.

25. The method of claim 15, wherein the altered amino acid residue is not Y361.

26. The method of claim 25, wherein the altered amino acid residue is not Y361L.

27. The method of claim 25, wherein the altered amino acid residue is not Y361C.

28. The method of claim 15, wherein the detection of the at least one variance comprises amplifying a segment of nucleic acid.

29. The method of claim 15, wherein the detection of the at least one variance comprises polony genotyping.

30. (canceled)

31. (canceled)

32. (canceled)

33. (canceled)

34. (canceled)

35. (canceled)

36. (canceled)

37. (canceled)

38. (canceled)

39. (canceled)

40. A method for selecting a chemotherapeutic drug to treat a patient with cancer, comprising:
(a) determining the level of FTI resistance in one or more cultured or biopsied cancer cells obtained from said patient according to the method of claim 15; and
(b) selecting a chemotherapeutic drug(s) to treat said patient based upon the level of FTI resistance in said patient's cancer cells, wherein the chemotherapeutic drug(s) can comprise a FTI if the patient's cancer cells have a relatively low level of FTI resistance, and the chemotherapeutic drug(s) do not comprise a FTI or comprise a relatively low of FTI if the patient's cancer cells have a relatively high level of FTI resistance.


SEQUENCE LISTING (61 sequences)

SEQ ID NO: 1 - DNA, 24 bp, Artificial Sequence
  Description of Artificial Sequence Synthetic primer
atggcttctccgagttctttcacc

SEQ ID NO: 2 - DNA, 25 bp, Artificial Sequence
  Description of Artificial Sequence Synthetic primer
tctcgagtcctctagtcggttgcag

SEQ ID NO: 3 - DNA, 20 bp, Artificial Sequence
  Description of Artificial Sequence Synthetic primer
ttttctctcctgtctctctc

SEQ ID NO: 4 - DNA, 20 bp, Artificial Sequence
  Description of Artificial Sequence Synthetic primer
cttgtctctcagagttgatg

SEQ ID NO: 5 - DNA, 20 bp, Artificial Sequence
  Description of Artificial Sequence Synthetic primer
tcactgagcctcattagctc

SEQ ID NO: 6 - DNA, 20 bp, Artificial Sequence
  Description of Artificial Sequence Synthetic primer
ttctgaagtagtgtcgtgac

SEQ ID NO: 7 - DNA, 20 bp, Artificial Sequence
  Description of Artificial Sequence Synthetic primer
ttgtgtacgtccactcactg

SEQ ID NO: 8 - DNA, 20 bp, Artificial Sequence
  Description of Artificial Sequence Synthetic primer
aagacagagcagctgctcac

SEQ ID NO: 9 - DNA, 20 bp, Artificial Sequence
  Description of Artificial Sequence Synthetic primer
tgcttcactctgtgtctatg

SEQ ID NO: 10 - DNA, 20 bp, Artificial Sequence
  Description of Artificial Sequence Synthetic primer
atccaggatagacagagctc

SEQ ID NO: 11 - DNA, 23 bp, Artificial Sequence
  Description of Artificial Sequence Synthetic primer
agggctggaggatggggctttta

SEQ ID NO: 12 - DNA, 23 bp, Artificial Sequence
  Description of Artificial Sequence Synthetic primer
gcatggctgcagtgctatcacga

SEQ ID NO: 13 - DNA, 21 bp, Artificial Sequence
  Description of Artificial Sequence Synthetic primer
atgggcttccatccctggtat

SEQ ID NO: 14 - DNA, 21 bp, Artificial Sequence
  Description of Artificial Sequence Synthetic primer
gctgctactccttctggcaga

SEQ ID NO: 15 - DNA, 24 bp, Artificial Sequence
  Description of Artificial Sequence Synthetic primer
cctggcaagtcgcgtgatttctta

SEQ ID NO: 16 - DNA, 27 bp, Artificial Sequence
  Description of Artificial Sequence Synthetic primer
gatgcctatgagcgtctggatgccagc

SEQ ID NO: 17 - DNA, 27 bp, Artificial Sequence
  Description of Artificial Sequence Synthetic primer
gctggcatccagacgctcataggcatc

SEQ ID NO: 18 - DNA, 23 bp, Artificial Sequence
  Description of Artificial Sequence Synthetic primer
ggctctgctataggatcctgcac

SEQ ID NO: 19 - DNA, 23 bp, Artificial Sequence
  Description of Artificial Sequence Synthetic primer
gtgcaggatcctatagcagagcc

SEQ ID NO: 20 - DNA, 27 bp, Artificial Sequence
  Description of Artificial Sequence Synthetic primer
ccacaccttgcatccacatatgcagca

SEQ ID NO: 21 - DNA, 27 bp, Artificial Sequence
  Description of Artificial Sequence Synthetic primer
tgctgcatatgtggatgcaaggtgtgg

SEQ ID NO: 22 - DNA, 25 bp, Artificial Sequence
  Description of Artificial Sequence Synthetic primer
gcacccacatattcagcagtcaatg

SEQ ID NO: 23 - DNA, 25 bp, Artificial Sequence
  Description of Artificial Sequence Synthetic primer
cattgactgctgaatatgtgggtgc

SEQ ID NO: 24 - DNA, 30 bp, Artificial Sequence
  Description of Artificial Sequence Synthetic primer
ctccgtagcctcgccgaccaacatcatcac

SEQ ID NO: 25 - DNA, 30 bp, Artificial Sequence
  Description of Artificial Sequence Synthetic primer
gtgatgatgttggtcggcgaggctacggag

SEQ ID NO: 26 - DNA, 27 bp, Artificial Sequence
  Description of Artificial Sequence Synthetic primer
gacctctttgagagcactgctgaatgg

SEQ ID NO: 27 - DNA, 27 bp, Artificial Sequence
  Description of Artificial Sequence Synthetic primer
ccattcagcagtgctctcaaagaggtc

SEQ ID NO: 28 - DNA, 27 bp, Artificial Sequence
  Description of Artificial Sequence Synthetic primer
ggtggcattggcgaggtaccagggatg

SEQ ID NO: 29 - DNA, 27 bp, Artificial Sequence
  Description of Artificial Sequence Synthetic primer
catccctggtacctcgccaatgccacc

SEQ ID NO: 30 - DNA, 27 bp, Artificial Sequence
  Description of Artificial Sequence Synthetic primer
ggcattggcgggataccagggatggaa

SEQ ID NO: 31 - DNA, 27 bp, Artificial Sequence
  Description of Artificial Sequence Synthetic primer
ttccatccctggtatcccgccaatgcc

SEQ ID NO: 32 - DNA, 29 bp, Artificial Sequence
  Description of Artificial Sequence Synthetic primer
taatcctcaagaggaaacgttccttgaac

SEQ ID NO: 33 - DNA, 29 bp, Artificial Sequence
  Description of Artificial Sequence Synthetic primer
gttcaaggaacgtttcctcttgaggatta

SEQ ID NO: 34 - DNA, 27 bp, Artificial Sequence
  Description of Artificial Sequence Synthetic primer
acaagccggcaggtgcgatttgaagga

SEQ ID NO: 35 - DNA, 27 bp, Artificial Sequence
  Description of Artificial Sequence Synthetic primer
tccttcaaatcgcacctgccggcttgt

SEQ ID NO: 36 - DNA, 29 bp, Artificial Sequence
  Description of Artificial Sequence Synthetic primer
gcagatgcgatttaaaggaggatttcagg

SEQ ID NO: 37 - DNA, 29 bp, Artificial Sequence
  Description of Artificial Sequence Synthetic primer
cctgaaatcctcctttaaatcgcatctgc

SEQ ID NO: 38 - DNA, 25 bp, Artificial Sequence
  Description of Artificial Sequence Synthetic primer
tccttctggcagacggggctcctgc

SEQ ID NO: 39 - DNA, 25 bp, Artificial Sequence
  Description of Artificial Sequence Synthetic primer
gcaggagccccgtctgccagaagga

SEQ ID NO: 40 - DNA, 27 bp, Artificial Sequence
  Description of Artificial Sequence Synthetic primer
aagtcgcgtgattcctaccacacctgc

SEQ ID NO: 41 - DNA, 27 bp, Artificial Sequence
  Description of Artificial Sequence Synthetic primer
gcaggtgtggtaggaatcacgcgactt

SEQ ID NO: 42 - DNA, 27 bp, Artificial Sequence
  Description of Artificial Sequence Synthetic primer
tcgcgtgatttcttacacacctgctac

SEQ ID NO: 43 - DNA, 27 bp, Artificial Sequence
  Description of Artificial Sequence Synthetic primer
gtagcaggtgtgtaagaaatcacgcga

SEQ ID NO: 44 - DNA, 27 bp, Artificial Sequence
  Description of Artificial Sequence Synthetic primer
tcgcgtgatttccaccacacctgctac

SEQ ID NO: 45 - DNA, 27 bp, Artificial Sequence
  Description of Artificial Sequence Synthetic primer
gtagcaggtgtggtggaaatcacgcga

SEQ ID NO: 46 - DNA, 26 bp, Artificial Sequence
  Description of Artificial Sequence Synthetic primer
cgcgtgatttctcccacacctgctac

SEQ ID NO: 47 - DNA, 26 bp, Artificial Sequence
  Description of Artificial Sequence Synthetic primer
gtagcaggtgtgggagaaatcacgcg

SEQ ID NO: 48 - protein, 4 aa, Artificial Sequence
  Description of Artificial Sequence Synthetic peptide
CVIM

SEQ ID NO: 49 - protein, 4 aa, Artificial Sequence
  Description of Artificial Sequence Synthetic peptide
CSIM

SEQ ID NO: 50 - protein, 4 aa, Artificial Sequence
  Description of Artificial Sequence Synthetic peptide
CAIM

SEQ ID NO: 51 - protein, 10 aa, Artificial Sequence
  Description of Artificial Sequence Synthetic peptide
KKSKTKCVIM

SEQ ID NO: 52 - protein, 6 aa, Artificial Sequence
  Description of Artificial Sequence Synthetic peptide
TKCVIM

SEQ ID NO: 53 - protein, 10 aa, Artificial Sequence
  Description of Artificial Sequence Synthetic peptide
RASNRSCAIM

SEQ ID NO: 54 - protein, 10 aa, Artificial Sequence
  Description of Artificial Sequence Synthetic peptide
TQSPQNCSIM

SEQ ID NO: 55 - protein, 4 aa, Artificial Sequence
  Description of Artificial Sequence Synthetic peptide
CIIM

SEQ ID NO: 56 - protein, 4 aa, Artificial Sequence
  Description of Artificial Sequence Synthetic peptide
CVVM

SEQ ID NO: 57 - protein, 4 aa, Artificial Sequence
  Description of Artificial Sequence Synthetic peptide
CVLS

SEQ ID NO: 58 - DNA, 23 bp, Artificial Sequence
  Description of Artificial Sequence Synthetic oligonucleotide
cacccccaccgccctcaaagtag

SEQ ID NO: 59 - DNA, 24 bp, Artificial Sequence
  Description of Artificial Sequence Synthetic oligonucleotide
tagttcctcaccttgtcgtattat

SEQ ID NO: 60 - DNA, 17 bp, Artificial Sequence
  Description of Artificial Sequence Synthetic primer
ctttatccagccctcac

SEQ ID NO: 61 - DNA, 21 bp, Artificial Sequence
  Description of Artificial Sequence Synthetic primer
accctaactgacacacattcc